(19) United States
(12) Patent Application Publication
(10) Pub. No.: US 2011/0040105 A1
(43) Pub. Date: Feb. 17, 2011

(54) PROCESSES USEFUL FOR THE SYNTHESIS OF (R)-1--2-METHYL-PYRROLIDINE

(75) Inventors: Christian H. Huber, San Diego, CA (US); Young Mi Khulman, San Diego, CA (US); Sagun K. Tandel, San Diego, CA (US); Stephen R. Johannsen, San Diego, CA (US); Tingmin Wang, San Diego, CA (US); Paul Angell, Carlsbad, CA (US)

Correspondence Address: FISH & RICHARDSON P.C., P.O. BOX 1022, MINNEAPOLIS, MN, 55440-1022, US

(73) Assignee: ARENA PHARMACEUTICALS, INC., San Diego, CA (US)

(21) Appl. No.: 12/988,109

(22) PCT Filed: Apr. 15, 2009
(86) PCT No.: PCT/US09/02333

§ 371 Date: Oct. 15, 2010

Related U.S. Application Data
(60) Provisional application No. 61/124,481, filed on Apr. 16, 2008.

(51) Int. Cl. C07D 295/023; C07C 315/04

(52) U.S. Cl. 548/579; 562/429

(57) ABSTRACT

Processes useful for making a pharmaceutically useful compound according to Formula (I), forms of such a compound, and intermediates useful in such processes are described.

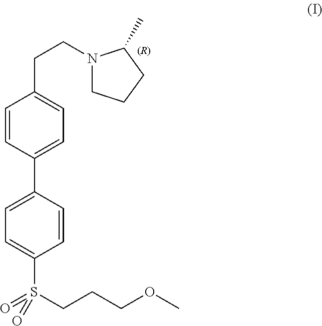

### TECHNICAL FIELD

The present invention relates to methods useful in the synthesis of an organic compound and salts thereof which are useful for the treatment of histamine H3-receptor associated disorders.

### BACKGROUND

The compound, (R)-1-{2-[4′-(3-methoxy-propane-1-sulfonyl)-biphenyl-4-yl]-ethyl}-2-methyl-pyrrolidine (the compound of formula I, below), which is described in PCT Application PCT/US2007/022086, which is incorporated herein by reference in its entirety, belongs to a class of histamine H3-receptor modulators that are useful in the treatment of histamine H3-receptor associated diseases and disorders.

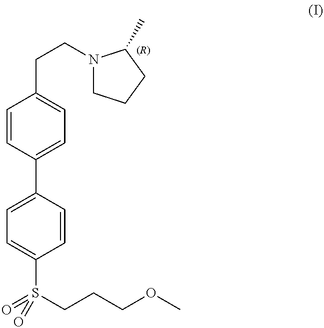

Efficient synthetic procedures are very important in the development of new drug compounds, both for providing economic routes to such compounds, as well as for the preparation of drug product that is pure and/or free of harmful contaminants. The synthetic procedures and intermediates described herein meet one or more of these and other needs.

### SUMMARY

In one aspect, a process is provided for preparing a compound according to formula I:

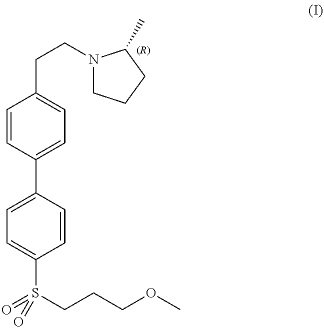

or a salt thereof, comprising reacting a compound according to formula II:

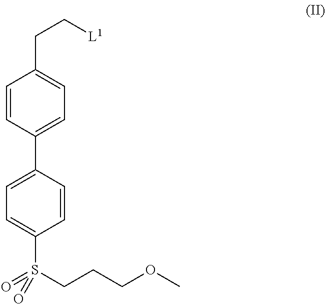

wherein L¹ is a suitable leaving group selected from iodide and a sulfonate ester group with a compound according to formula III:

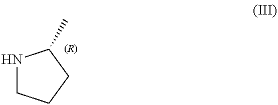

or a salt thereof, under conditions sufficient to effect displacement of the leaving group L¹ of the compound according to formula II by the amino group of the compound according to formula III to form the compound according to formula I, or a salt thereof.

In some embodiments thereof, the process further comprises a step, wherein the compound according to formula II is prepared by a process comprising reacting a compound according to formula IV:

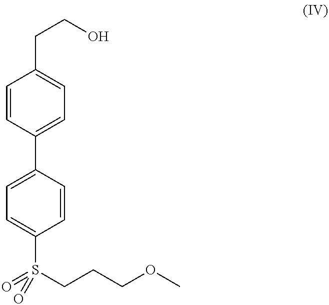

under conditions sufficient to effect conversion of the hydroxyl group to form the leaving group L¹ of the compound according to formula II.

In some embodiments thereof, the process further comprises a step, wherein the compound according to formula IV is prepared by a process comprising reducing a compound according to formula V:

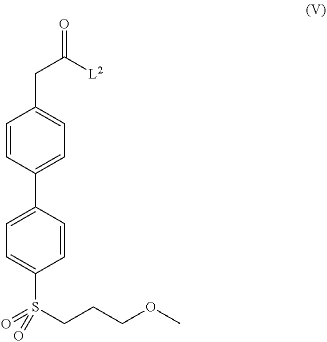

wherein L² is hydroxyl, or a salt of the hydroxyl, or L² is C₁-C₆ alkoxy.

In some embodiments thereof, the process further comprises a step, wherein the compound according to formula V is prepared by a process comprising reacting a compound according to formula VI:

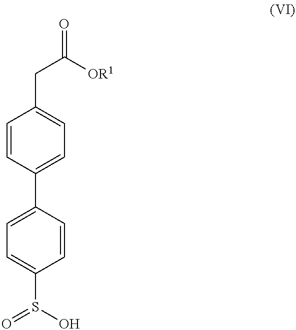

or salt thereof, wherein R¹ is hydrogen or C₁-C₆ alkyl, with a compound according to formula VII:

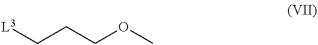

wherein L³ is a suitable leaving group, under conditions sufficient to effect displacement of the leaving group L³ of the compound according to formula VII by the sulfinate group of the compound according to formula VI.

In some embodiments thereof, the process further comprises a step, wherein the compound according to formula VI is prepared by a process comprising reducing a compound according to formula VIII:

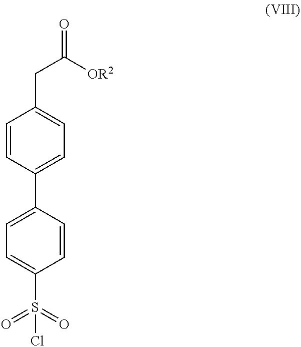

or a salt thereof, wherein R² is hydrogen or C₁-C₆ alkyl.

In some embodiments thereof, the process further comprises a step, wherein the compound according to formula VIII is prepared by a process comprising chlorosulfonating a compound according to formula IX:

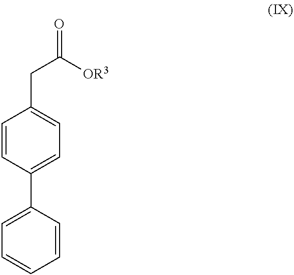

wherein R³ is hydrogen or R³ or C₁-C₆ alkyl.

In another aspect there is provided a process for preparing a compound according to formula I:

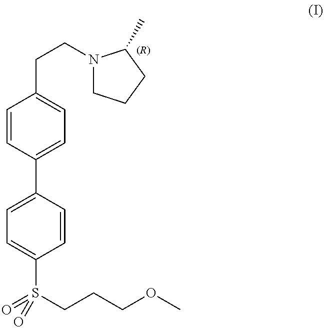

or a salt thereof, comprising providing a starting material according to formula IX:

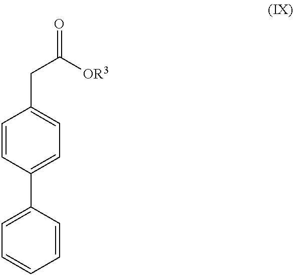

or a salt thereof, wherein R³ is hydrogen or C₁-C₆ alkyl, and performing a reaction sequence comprising:
(a) performing a chlorosulfonation reaction and reducing the resulting sulfonyl chloride to form a sulfinate salt and alkylating the sulfinate salt with an alkylating agent according to formula VII:

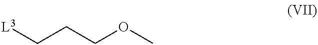

(b) converting the optionally protected carboxyl group —C(═O)OR3 to form a group according to the formula X:

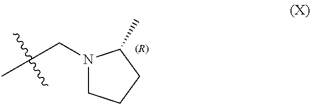

to form the compound according to formula I, or a salt thereof.

In further aspects, there are provided processes useful in the synthesis of compounds according to formula I, and in preparing intermediates useful in such processes. A process is provided for preparing a compound according to formula II:

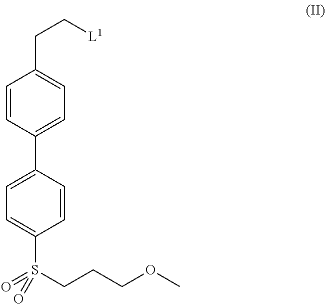

wherein L¹ is a leaving group selected from iodide and a sulfonate ester group, comprising reacting a compound according to formula IV:

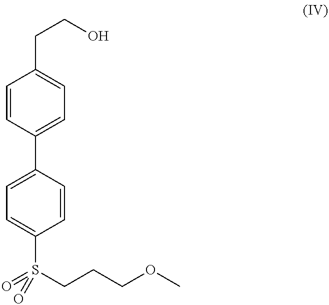

under conditions sufficient to effect conversion of the hydroxyl group to form the leaving group L¹ of the compound according to formula II.

A process is provided for preparing a compound according to formula IV:

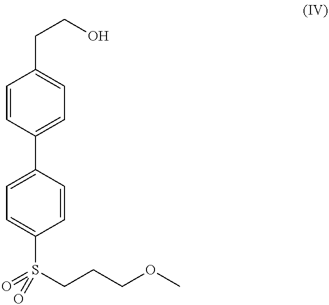

comprising reducing a compound according to formula V:

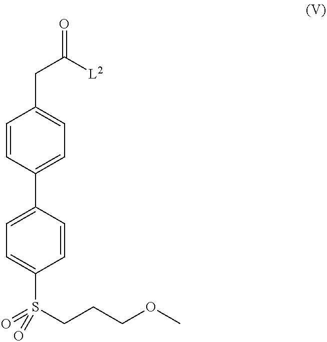

wherein L² is hydroxyl, or a salt of the hydroxyl, or L² is C₁-C₆ alkoxy.

In another aspect there is provided a process for preparing a compound according to formula V:

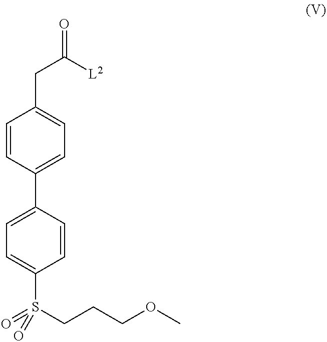

wherein L² is hydroxyl, or a salt of the hydroxyl, or L² is C₁-C₆ alkoxy, comprising reacting a compound according to formula VI:

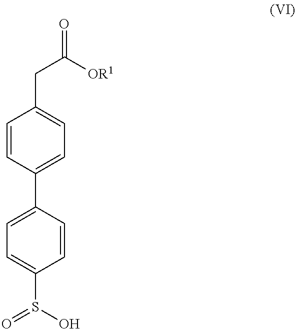

or salt thereof, wherein R¹ is hydrogen or C₁-C₆ alkyl, with a compound according to formula VII:

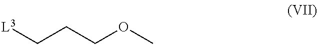

wherein L³ is a suitable leaving group, under conditions sufficient to effect displacement of the leaving group L³ of the compound according to formula VII by the sulfinate group of the compound according to formula VI.

A process is provided for preparing a compound according to formula VI:

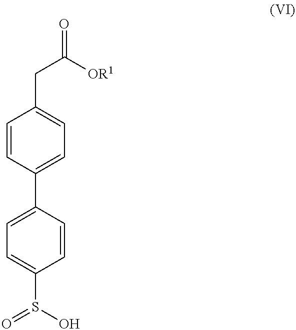

or a salt thereof, wherein R¹ is hydrogen or C₁-C₆ alkyl, comprising reducing a compound according to formula VIII:

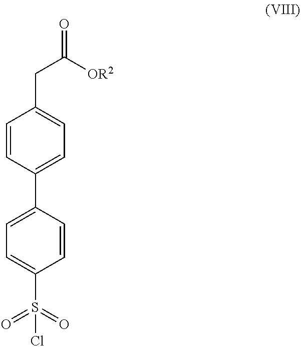

or a salt thereof, wherein R² is hydrogen or C₁-C₆ alkyl.

A process is provided for preparing a compound according to formula VIII:

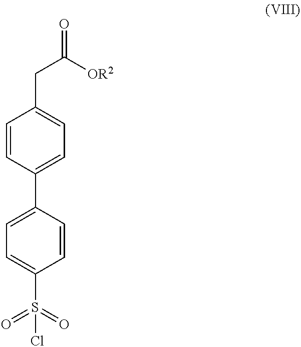

or a salt thereof, wherein R² is hydrogen or C₁-C₆ alkyl, comprising chlorosulfonating a compound according to formula IX:

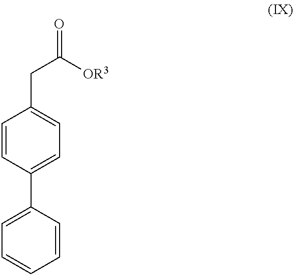

or a salt thereof, wherein R³ is hydrogen or C₁-C₆ alkyl.

Also provided are processes for preparing salts of a compound according to formula I:

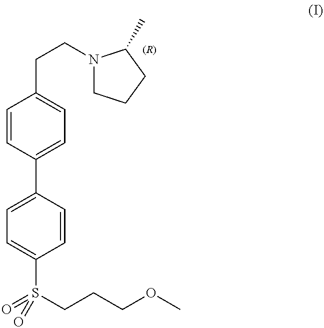

comprising reacting a compound according to formula I with an acid, for example citric acid, for example in a solvent other than acetonitrile.

In other aspects, there are provided novel and useful intermediates useful in the synthesis of a compound according to formula I.

As one aspect there is provided a compound according to formula XI:

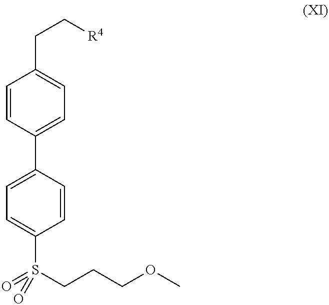

wherein R⁴ is iodide, hydroxyl, or a sulfonate ester.

Also provided is a compound according to formula V:

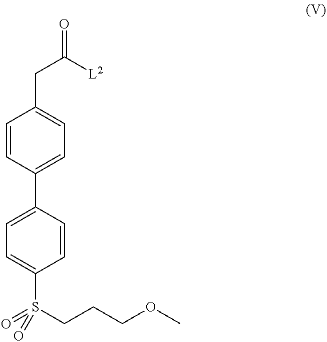

wherein L² is hydroxyl, or a salt of the hydroxyl, or L² is C₁-C₆ alkoxy.

Also provided is a compound according to formula VI:

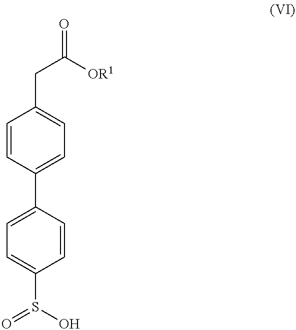

or a salt thereof, wherein R¹ is hydrogen or C₁-C₆ alkyl.

Also provided is a compound according to formula VIII:

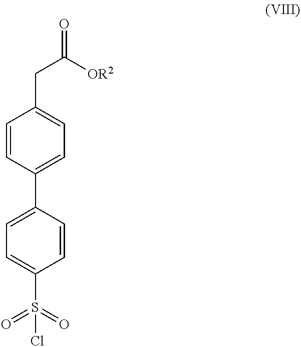

or a salt thereof, wherein R² is hydrogen or C₁-C₆ alkyl.

### DETAILED DESCRIPTION

The present application provides methods of synthesis of (R)-1-{2-[4′-(3-methoxypropane-1-sulfonyl)-biphenyl-4-yl]-ethyl}-2-methyl-pyrrolidine, and salts, and compositions thereof that modulate the activity of the histamine H3-receptor and are useful in the treatment of histamine H3-receptor associated disorders, such as, cognitive disorders, epilepsy, brain trauma, depression, obesity, disorders of sleep and wakefulness such as narcolepsy, shift-work syndrome, drowsiness as a side effect from a medication, maintenance of vigilance to aid in completion of tasks and the like, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, pain, dementia, Alzheimer's disease and the like. Also provided are intermediates useful in the synthesis of such compounds.

### I. Definitions

As used herein, the singular forms “a,” “an” and “the” include plural referents unless the context clearly dictates otherwise.

“Leaving group” means a univalent group (—X) which, when attached to hydrogen, is an acid (H-X) with a pKa of about 5 or lower, or, in the case of preferred leaving groups, a pKa of about 2 or lower. Thus, a leaving group is a functional group of a compound that in a nucleophilic substitution may be displaced to give, typically, a stable anion. Examples of leaving groups include halogen, for example chloride, bromide, and iodide, and sulfonate ester groups, for example trifluoromethanesulfonate (—OTf), arenesulfonates (such as phenylsulfonate, p-toluenesulfonate (—OTs), and naphthalenesulfonate), or alkanesulfonates (such as mesylate). The term “(Cₓ-C_{y})alkyl” (wherein x and y are integers) refers to an alkyl group containing between x and y carbon atoms. An alkyl group formally corresponds to an alkane with one C—H bond replaced by the point of attachment of the alkyl group to the remainder of the compound. An alkyl group may be straight-chained or branched. Alkyl groups having 3 or more carbon atoms may be cyclic. Cyclic alkyl groups having 7 or more carbon atoms may contain more than one ring and be polycyclic. Examples of straight-chained alkyl groups include methyl, ethyl, n-propyl, n-butyl, and n-octyl. Examples of branched alkyl groups include i-propyl, t-butyl, and 2,2-dimethylethyl. Examples of cyclic alkyl groups include cyclopropyl, cyclobutyl, cyclopentyl, cyclohexyl, cyclohexylmethyl, and 4-methylcyclohexyl. Examples of polycyclic alkyl groups include bicyclo[2.2.1]heptanyl, norbornyl, and adamantyl.

The term “(Cₓ-C_{y})alkoxy” (wherein x and y are integers) means a (Cₓ-C_{y})alkyl radical, as defined herein, attached directly to an oxygen atom. Examples include methoxy, ethoxy, n-propoxy, iso-propoxy, n-butoxy, t-butoxy, iso-butoxy, sec-butoxy and the like.

The term “Cₓ-C_{y} alkanonitrile” (wherein x and y are integers) means a compound of formula Alk-C≡N where Alk represents an alkyl group and the compound has between x and y carbon atoms (including the carbon atom of the nitrile group). Examples include acetonitrile, propionitrile, and butyronitrile.

The term “Cₓ-C_{y} alkanone” (wherein x and y are integers) means a compound of formula Alk-(C═O)-Alk′ where Alk and Alk′ each represents an independently selected alkyl group and the compound has between x and y carbon atoms (including that of the carbonyl group). Examples include acetone, 2-butanone and 2- and 3-pentanone.

The term “Cₓ-C_{y} alkanol” (wherein x and y are integers) means a compound of formula Alk-OH where Alk represents an alkyl group and the compound has between x and y carbon atoms. Examples include methanol, ethanol, n-propanol, isopropanol, n-butanol, and t-butanol.

The term “aliphatic ether” means a compound which is formally an alkane wherein an oxygen atom has been inserted into one or more C—C bonds to replace the C—C bonds with one or more ether groups. Examples are acyclic ethers, for example diethyl ether, diisopropyl ether, methyl t-butyl ether, and 1,2-dimethoxyethane, and cyclic ethers, for example tetrahydrofuran, 2-methyltetrahydrofuran and 1,4-dioxane.

The term “carboxylic acid solvent” means a C₁-C₆ alkanoic acid (i.e. a compound of the formula Alk-(C═O)OH wherein Alk is an alkyl group) the alkyl group of which is optionally substituted with fluorine. Examples include acetic acid, propionic acid, butyric acid, trifluoroacetic acid, and pentafluoropropionic acid.

A “protecting group” is a derivative of a chemical functional group that is stable to some reaction conditions but may be removed under other conditions, where general types of conditions under which the group will be stable and may be removed are known to the person skilled in the art. This property makes it possible to perform reactions where a functional group would otherwise be incompatible with the conditions required to perform a particular reaction if a protecting group is used which is stable under the conditions, but which can subsequently be removed to regenerate the original functional group, which can thereby considered to have been “protected”. Protecting groups may also be used for other purposes (e.g. where the “protected” derivative is more soluble or easier to purify than the compound having an “unprotected” functional group). The person skilled in the art knows when protecting groups may be useful, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis by Theodora W. Greene, Peter G. M. Wuts, John Wiley & Sons Ltd (3^{rd} Ed., 1999) (“Greene”), the entire disclosure of which is incorporated herein by reference. Of particular interest in the context of the present invention are protecting groups of carboxyl groups, which are described in Chapter 5 of Greene, and which esters are of particular interest, which include methyl esters, substituted methyl esters (e.g. methoxymethyl, methylthiomethyl, tetrahydropyranyl, tetrahydrofuranyl, methoxyethoxymethyl, 2-trimethylsilylethoxymethyl, benzyloxymethyl), ethyl, substituted ethyl esters (e.g. 2,2,2-trichloroethyl, 2-trimethylsilylethyl, 2-cyanoethyl), n-alkyl (e.g. n-propyl, n-butyl, n-pentyl), branched alkyl (e.g. isopropyl, t-butyl), allyl, phenyl, benzyl, substituted benzyl (e.g., triphenylmethyl, p-bromobenzyl) etc.

### II. Chemical Processes

The inventors have discovered that (R)-1-{2-[4′-(3-methoxy-propane-1-sulfonyl)-biphenyl-4-yl]-ethyl}-2-methyl-pyrrolidine may be efficiently synthesized starting from 4-biphenylacetic acid (the compound of formula IX wherein R³═H) (or a derivative thereof) a compound which is commercially available, or a protected derivative thereof, by a reaction scheme which is illustrated in Scheme 1 below.

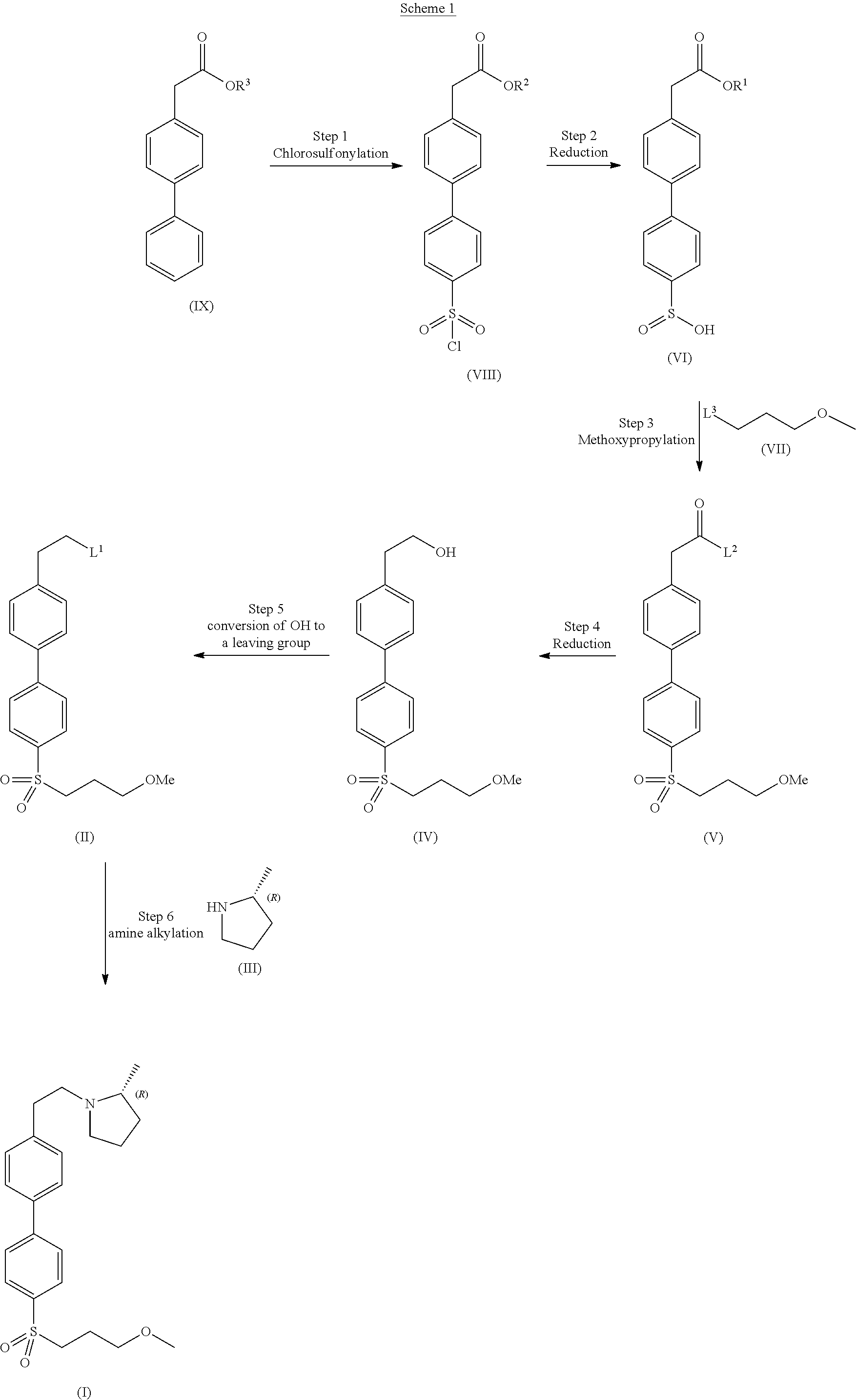

The process comprises providing 4-biphenylacetic acid (the compound of formula IX wherein R³═H) (or a protected derivative thereof) and elaborating the acetic acid group to provide the (R)-(2-methylpyrrolidinyl)ethyl group, and at the 4′-position performing a chlorosulfonation reaction, reducing the resulting sulfonyl chloride, and introducing the 3-methoxypropyl group of the compound according to formula I by alkylation.

Accordingly, in general terms, there is provided a process for preparing a compound according to formula I:

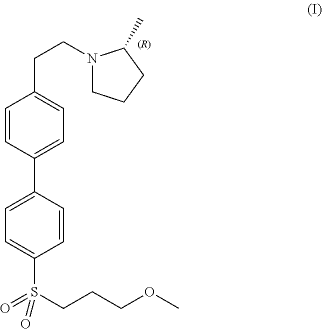

or a salt thereof, comprising providing a starting material according to formula IX:

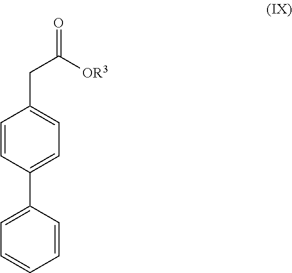

or a salt thereof, wherein R³ is hydrogen or C₁-C₆ alkyl, and performing a reaction sequence comprising:
(a) performing a chlorosulfonation reaction and reducing the resulting sulfonyl chloride to form a sulfinate salt and alkylating the sulfinate salt with an alkylating agent according to formula VII:

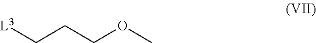

(b) converting the optionally protected carboxyl group —C(═O)OR3 to form a group according to the formula X:

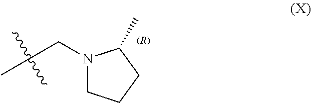

to form the compound according to formula I, or a salt thereof.

In some embodiments of such a process, R³ is hydrogen.

One aspect of the present invention pertains to a process for preparing a compound according to formula I:

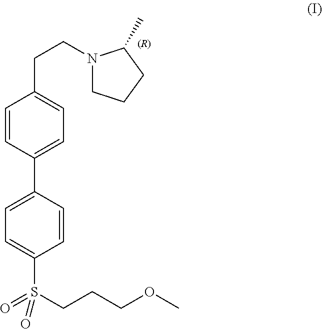

or a salt thereof, comprising:
(a) chlorosulfonating a compound according to formula IX:

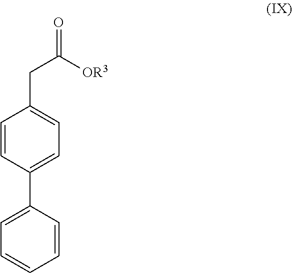

or a salt thereof, to form a compound according to formula VIII:

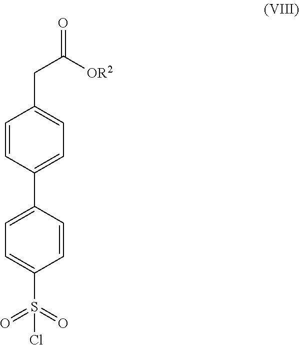

or a salt thereof;
(b) reducing the compound according to formula VIII or a salt thereof, to form a compound according to formula VI:

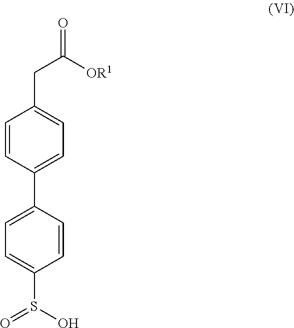

or a salt thereof;
(c) reacting the compound according to formula VI or salt thereof, with a compound according to formula VII:

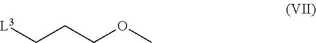

under conditions sufficient to effect displacement of the leaving group L³ of the compound according to formula VII by the sulfinate group of the compound according to formula VI to form a compound according to formula V:

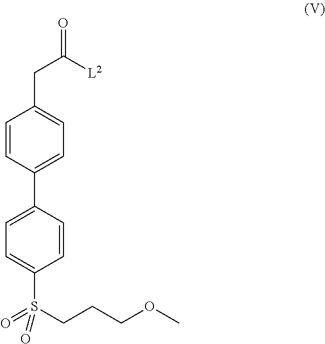

(d) reducing the compound according to formula V to form a compound according to formula IV:

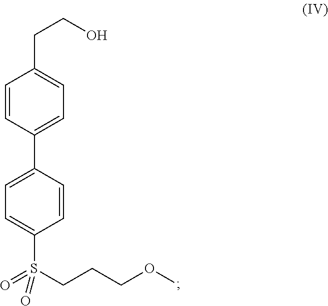

(e) reacting the compound according to formula IV to form a compound according to formula II:

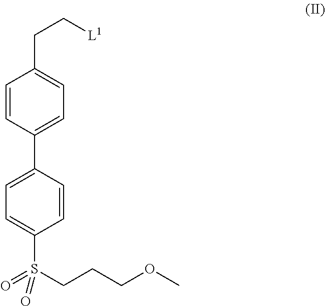

under conditions sufficient to effect conversion of the hydroxyl group of the compound according to formula IV to form the leaving group L¹ of the compound according to formula II; and
(f) reacting the compound according to formula II with a compound according to formula III:

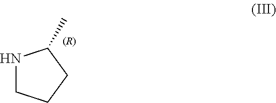

or a salt thereof, under conditions sufficient to effect displacement of the leaving group L¹ of the compound according to formula H by the amino group of the compound according to formula III to form the compound according to formula I, or a salt thereof; wherein:
   L¹ is a suitable leaving group selected from iodide and a sulfonate ester group;
   L² is hydroxyl, or a salt of the hydroxyl, or L² is C₁-C₆ alkoxy
   L³ is a suitable leaving group;
   R¹ is hydrogen or C₁-C₆ alkyl;
   R² is hydrogen or C₁-C₆ alkyl; and
   R³ is hydrogen or C₁-C₆ alkyl.

The compound according to formula I optionally may be converted to a salt, for example following its synthesis by the methods described herein. Accordingly, in some embodiments of such a process the method further comprises reacting the compound according to formula I with an acid and isolating a salt of the compound according to formula I. In some embodiments, the salt is a citrate. In some sub-embodiments thereof, the salt is a mono-citrate. In other embodiments, the salt is a di-citrate. In some embodiments, the salt is a maleate. In some embodiments, the salt is a hydrochloride. Methods for salt formation are discussed in detail below.

### 1. Amine Alkylation Step (Step 6)

In one aspect a process is provided for preparing a compound according to formula I:

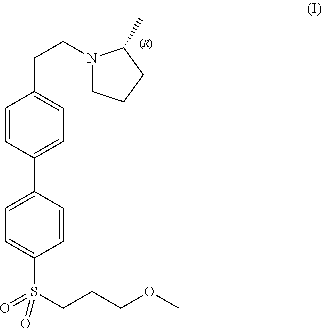

or a salt thereof, comprising reacting a compound according to formula II:

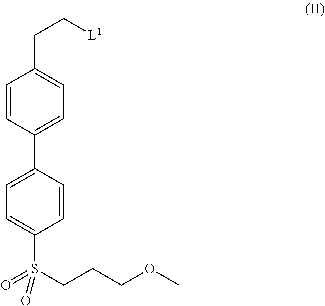

wherein L¹ is a suitable leaving group selected from chloride, bromide, iodide and a sulfonate ester group with a compound according to formula III:

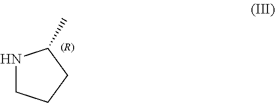

or a salt thereof, under conditions sufficient to effect displacement of the leaving group L¹ of the compound according to formula II by the amino group of the compound according to formula III to form the compound according to formula I, or a salt thereof.

Any suitable leaving group may be used as L¹ in the aforementioned process. Suitable leaving groups include halogen, for example chloride, bromide, or iodide, and sulfonate ester groups, for example, trifluoromethanesulfonate (—OTf), arenesulfonates (such as phenylsulfonate, p-toluenesulfonate (—OTs), and naphthalenesulfonate), or alkanesulfonates (such as mesylate).

In some embodiments, L¹ is a halogen selected from chloride, bromide, and iodide, or a sulfonate ester group.

In some embodiments, L¹ is iodide, or a sulfonate ester group. In some embodiments, L¹ is a sulfonate ester group. In some embodiments, L¹ is a methanesulfonate ester group. The compound according to formula III ((R)-2-methylpyrrolidine) is also commercially available, or it may be made by methods known to one skilled in the art, for example by the reduction of suitable proline derivatives (see, e.g., D. Zhao, et al., “Efficient and Practical Synthesis of (R)-2-Methylpyrrolidine”, J. Org. Chem., 2006, 71 (11), 4336-38). The compound according to formula III may be used in the reaction in the form of the free base or in the form of a salt. In some embodiments, the compound according to formula III in the form of a salt, for example, a tartrate salt such as the L-tartrate salt.

Any relative amounts of the compounds of formulae II and III may be used to convert the compound according to formula II to provide the compound according to I (with the extent of conversion dependent on the amount of the compound according to formula III used). It is believed that relative molar amounts of the compounds II and III used in the process should optimally be close to about 1:1 with the use of a modest excess of the compound of formula III being beneficial to ensure complete and reasonable conversion of the compound according to formula II. The molar ratio of the compound according to formula III to that of the compound of formula II used in the process is beneficially in the range from about 0.8:1 to about 3:1, such as at least about 1:1, or at least about 1.1:1. For example, molar ratios in the range from about 1:1 to about 3:1, about 1.1:1 to about 3:1, about 1:1 to about 2:1, about 1.1:1 to about 2:1, about 1:1 to about 1.5:1, or about 1.1:1 to about 1.5:1 are suitable. An example of a suitable ratio is about 1.4:1.

In some embodiments, the reacting is performed in the presence of a suitable base. In some embodiments, the base is an alkali metal carbonate. When the compound according to formula III is employed in the form of a salt, the base liberates the free base form of the compound of formula III. The amount of base that may be used is at least about one equivalent relative to the compound according to formula II. Suitable bases include organic bases, such as tertiary amine bases, particularly hindered tertiary amine bases, for example triethylamine or N,N-diisopropylethylamine, and inorganic bases such as alkali metal or alkaline earth carbonates. Bases which may be used include alkali metal carbonates, for example sodium or potassium carbonate. In some embodiments, the base is an alkali metal carbonate. In some embodiments, the base is potassium carbonate.

Most solvents in which sufficient solubility of the reagents can be achieved should be suitable for performing the process described herein. In some embodiments, the reacting is performed in the presence of an aprotic solvent. In some embodiments, the aprotic solvent comprises a C₂-C₄ alkanonitrile. In some embodiments, the aprotic solvent comprises acetonitrile. In some embodiments, the solvent comprises a C₃-C₅ alkanone. In some embodiments, the C₃-C₅ alkanone is 2-butanone. In some embodiments, the reacting is performed in the presence of a solvent comprising water. In some embodiments, the reacting is performed in the presence of water. An example of a suitable solvent mixture is a mixture of acetonitrile and water in a ratio 8:3 by volume. A further example of a suitable solvent mixture is a mixture of 2-butanone and water in a ratio of about 8:3 by volume to about 8:2 by volume. A further example of a suitable solvent mixture is a mixture of 2-butanone and water in a ratio of about 8:3 by volume. A further example of a suitable solvent mixture is a mixture of 2-butanone and water in a ratio of about 8:2 by volume.

The reacting can be performed at ambient or elevated temperature. In some embodiments, the reacting is performed at a temperature in the range from about 30° C. to about 120° C.

In some embodiments, the reacting is performed at a temperature in the range from about 60° C. to about 80° C. In some embodiments, the reacting is performed at a temperature of about 70° C.

Progress of the reaction may be followed by standard analytical techniques, for example thin layer chromatography, or HPLC. The reaction can be allowed to continue until the conversion of the limiting reagent is at least about 80%, at least about 90%, at least about 95%, at least about 98%, or at least about 99% complete.

Following the synthesis of the compound of formula I, the compound according to formula I may optionally be converted to a salt. Accordingly, in some embodiments of such a process the method further comprises reacting the compound according to formula I with an acid and isolating a salt of the compound according to formula I. In some embodiments, the salt is a citrate. In some sub-embodiments thereof, the salt is a mono-citrate. In other embodiments, the salt is a di-citrate. Methods for salt formation are discussed in detail below.

In some embodiments of the process described herein, the process further comprises isolating a compound according to formula I, or a salt thereof, wherein the isolated compound according to formula I, or salt thereof, has a purity of at least about 80% by weight, at least about 90% by weight, at least about 95% by weight, at least about 98% by weight, or at least about 99% by weight.

In some embodiments of the process described herein, the process further comprises isolating a compound according to formula I, or a salt thereof, wherein the isolated compound according to formula I, or salt thereof, has an enantiomeric excess of at least about 80%, at least about 90%, at least about 95%, at least about 98%, or at least about 99%.

### 2. Preparation of the Compound According to Formula II by Conversion of the Hydroxyl of the Compound According to Formula IV to a Leaving Group (Step 5)

In another aspect there is provided a process for the preparation of a compound according to formula II:

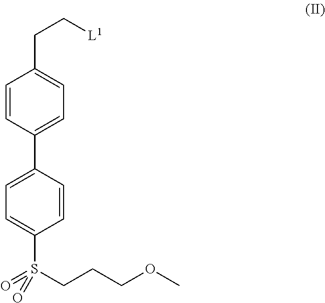

wherein L¹ is a leaving group selected from chloride, bromide, iodide and a sulfonate ester group, comprising reacting a compound according to formula IV:

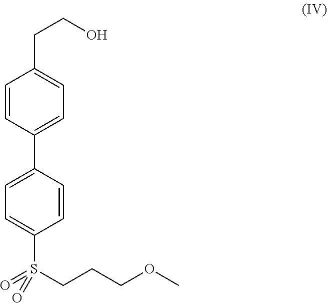

under conditions sufficient to effect conversion of the hydroxyl group to form the leaving group L¹ of the compound according to formula II.

In some embodiments, L¹ is iodide or a sulfonate ester group.

In some embodiments, L¹ is a chloride group. The process may be performed by reacting the compound according to formula IV with a suitable chlorinating agent, for example N-chlorosuccinimide or carbon tetrachloride and triphenylphosphine.

In some embodiments, L¹ is a bromide group. The process may be performed by reacting the compound according to formula IV with a suitable brominating agent , for example bromine, N-bromosuccinimide or carbon tetrabromide and triphenylphosphine.

In some embodiments, L¹ is an iodide group. The process may be performed by reacting the compound according to formula IV with a suitable iodinating agent, for example iodine and triphenylphosphine.

In other embodiments, L¹ is a sulfonate ester group. The process may be performed by reacting the compound according to formula IV with a suitable sulfonylating agent, for example a sulfonic acid derivative which can react electrophilically with the hydroxyl group of the compound according to formula IV to esterify the hydroxyl group as a sulfonate ester. Examples of suitable sulfonic acid derivatives are sulfonyl halides, such as the sulfonyl chloride, and sulfonic anhydrides.

In some embodiments, L¹ is a methanesulfonate ester group. In some embodiments, the compound according to formula II is prepared by reacting the compound according to formula IV with a methanesulfonylating agent. In some embodiments, the compound according to formula II is prepared by reacting the compound according to formula IV with methanesulfonyl chloride. In some embodiments, L¹ is a methanesulfonate ester group, and the compound according to formula II is prepared by reacting the compound according to formula IV with methanesulfonyl chloride. The process may be performed by reacting the compound according to formula IV with a methanesulfonylating agent, for example a methanesulfonyl halide, for example methanesulfonyl chloride, or methanesulfonic anhydride. A suitable methanesulfonylating agent is methanesulfonyl chloride.

The reagent used to effect the conversion of the hydroxyl group of the compound according to formula IV to the leaving group L¹, e.g. a sulfonylating agent such as methanesulfonyl chloride, can be used in excess relative to the amount of the compound according to formula IV. Therefore, it is believed that the molar ratio of the reagent (e.g. a sulfonylating agent, such as methanesulfonyl chloride) to that of the compound of formula II used in the process is beneficially in the range from about 0.8:1 to about 3:1, such as at least about 1:1, at least about 1.1:1, for example in the range from about 1:1 to about 3:1, about 1.1:1 to about 3:1, about 1:1 to about 2:1, about 1.1:1 to about 2:1, about 1:1 to about 1.5:1, or about 1.1:1 to about 1.5:1. An example of a suitable ratio is about 1.4:1.

In some embodiments, the reacting to form the compound according to formula II is performed in the presence of a base. In some embodiments, the base comprises a trialkylamine. In some embodiments, the base comprises N,N-diisopropylethylamine.

In some embodiments, the reacting to form the compound according to formula II is performed in an aprotic solvent. In some embodiments, the aprotic solvent comprises a C₂-C₄ alkanonitrile. In some embodiments, the aprotic solvent comprises acetonitrile. In some embodiments, the aprotic solvent comprises an aliphatic ether, a C₂-C₄ alkanonitrile, or a mixture thereof. In some embodiments, the aprotic solvent comprises a mixture of an aliphatic ether and a C₂-C₄ alkanonitrile. In some embodiments, the aliphatic ether is methyl t-butyl ether. In some embodiments, the C₂-C₄ alkanonitrile is acetonitrile. In some embodiments, for example, a methanesulfonylation reaction is performed in a reaction mixture wherein the solvent is a mixture of methyl t-butyl ether and acetonitrile in a ratio of about 4:1 by weight. In some embodiments, for example, a methanesulfonylation reaction is performed in a reaction mixture wherein the solvent is acetonitrile.

In some embodiments, the reacting to form the compound according to formula II is performed at about ambient temperature or lower. In some embodiments, the reacting to form the compound according to formula II is performed at a temperature in the range from about −20° C. to about 20° C. In some embodiments, the reacting to form the compound according to formula II is performed at a temperature in the range from about 0° C. to about 10° C.

The process described herein for the preparation of a compound according to formula II, or any of the embodiments thereof, may optionally be used for the synthesis of the compound according to formula II to be used in the aforementioned process for the synthesis of the compound according to formula I, or a salt thereof, or any of the embodiments of such a process.

### 3. Preparation of the Compound According to Formula IV by Reducing an Acid or Derivative Thereof According to Formula V (Step 4)

In another aspect there is provided a process for preparing a compound according to formula IV:

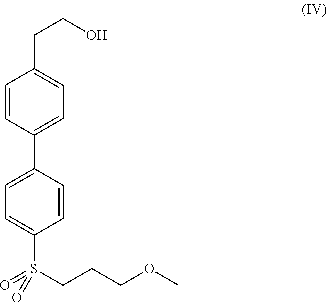

comprising reducing a compound according to formula V:

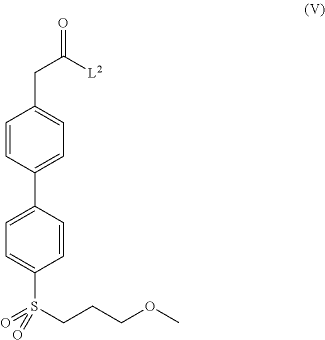

wherein L² is hydroxyl, or a salt of the hydroxyl, or L² is C₁-C₆ alkoxy.

Reducing the compound according to formula V can be performed directly using any of a wide variety of methods known in the art for reducing carboxylic acids or esters to alcohols. The reduction may be also performed indirectly, for example by converting the carboxylic acid or ester to another carboxylic acid derivative (such as an anhydride) and reducing that derivative, or by performing a step-wise reduction, e.g. reducing the compound according to formula V first to an aldehyde and reducing the aldehyde to the alcohol. In some embodiments, reducing the compound according to formula V is achieved by reacting the compound according to formula V with a suitable reducing agent. Examples of suitable reducing agents for the reduction of acids and esters include aluminium hydrides, e.g. lithium aluminium hydrides, and boron hydrides, for example borane. Lithium borohydride is effective as a reagent to reduce esters. Sodium borohydride may also be used in such reductions, although is generally not effective when used alone in the reduction of carboxylic acids. However, sodium borohydride used in conjunction with boron trifluoride is effective for the reduction of carboxylic acids wherein it is believed that the reaction of sodium borohydride with the boron trifluoride produces borane in situ. The boron trifluoride is generally used in the form of an etherate complex for such reactions.

In some embodiments, L² is hydroxyl or a salt of the hydroxyl, and the reducing agent for reducing the compound according to formula V comprises a boron hydride (a compound comprising boron-hydrogen bonds). In some embodiments thereof, the boron hydride is diborane (i.e. B₂H₆, which, when dissolved in a solvent may exist in the form of a solvent-BH₃ complex). In some embodiments, the boron hydride is diborane or a BH₃ complex. In some embodiments, the boron trifluoride used is in the form of a boron trifluoride etherate complex. In some embodiments, wherein L² is hydroxyl or a salt of the hydroxyl, wherein reducing the compound according to formula V is performed by reacting the compound with an alkali metal borohydride in the presence of boron trifluoride. In some embodiments, the alkali metal borohydride is sodium borohydride.

In some embodiments, reducing the compound according to formula V is performed in an aliphatic ether solvent. In some embodiments, the aliphatic ether solvent used in the reaction to form the compound according to formula IV is tetrahydrofuran. In some embodiments of the process wherein L² is hydroxyl, or a salt of the hydroxyl, reducing the compound according to formula V is performed by reacting the compound with an alkali metal borohydride, for example sodium borohydride, in the presence of a boron trifluoride.

In some embodiments, reducing the compound according to formula V is performed at about ambient temperature or lower. In some embodiments, reducing the compound according to formula V is performed at a temperature in the range from about −20° C. to about 30° C. In some embodiments, reducing the compound according to formula V is performed at a temperature in the range from about 0° C. to about 15° C.

The reagent used to effect the reduction of the compound according to formula V to the leaving group, e.g. diborane, may be used in excess relative to the amount of the compound according to formula V. For example, when sodium borohydride and boron trifluoride are used to generate the reducing agent, an example of a suitable amount of sodium borohydride and boron trifluoride is about 1.5 equivalents of each relative to the compound according to formula V.

The process described herein for the preparation of a compound according to formula IV, or any of the embodiments thereof, may optionally be used for the synthesis of the compound according to formula IV to be used in the aforementioned process for the synthesis of the compound according to formula II, or any of the embodiments of such a process, and which may further be used in the aforementioned process for the synthesis of the compound according to formula I, or a salt thereof, or any of the embodiments of such a process.

### 4. Preparation of the Compound According to Formula V by Alkylating a Sulfinic Acid According to Formula VI (Step 3)

In another aspect there is provided a process for preparing a compound according to formula V:

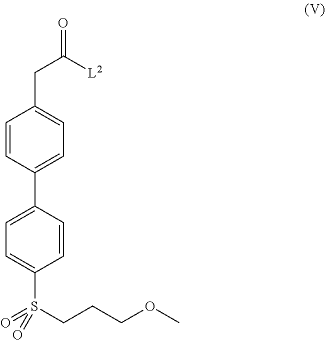

wherein L² is hydroxyl, or a salt of the hydroxyl, or L² is C₁-C₆ alkoxy, comprising reacting a compound according to formula VI:

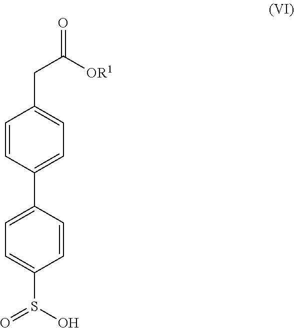

or salt thereof, wherein R¹ is hydrogen or C₁-C₆ alkyl, with a compound according to formula VII:

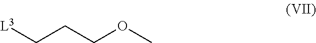

wherein L³ is a suitable leaving group, under conditions sufficient to effect displacement of the leaving group L³ of the compound according to formula VII by the sulfinate group of the compound according to formula VI.

Suitable compounds according to formula VII are known, commercially available, or may readily be prepared by methods known to one of ordinary skill in the art. Examples of suitable compounds according to Formula VII include those wherein L³ is chloride, bromide, iodide, or a sulfonate ester group, for example a methanesulfonate, benzenesulfonate, p-toluenesulfonate. In some embodiments, L³ is a bromide.

In some embodiments of the process, R¹ is hydrogen.

In some embodiments, the reacting to form the compound according to formula V is performed using an alkali metal salt of the compound according to formula VI. In some embodiments, the reacting to form the compound according to formula V is performed using a sodium salt or a di-sodium salt of the compound according to formula VI. In some embodiments, the reacting to form the compound according to formula V is performed using the di-sodium salt of 4′-sulfinobiphenyl-4-carboxylic acid (D).

In some embodiments, the reacting to form the compound according to formula V is performed in the presence of a catalyst. In some embodiments, the catalyst comprises a tetraalkylammonium salt. In some embodiments, the catalyst comprises an iodide salt. In some embodiments, the catalyst comprises tetra-n-butylammonium iodide. In some embodiments, the reacting to form the compound according to formula V is performed using the di-sodium salt of 4′-sulfinobiphenyl-4-carboxylic acid (D), in the presence of a solvent comprising water. In some embodiments, the reacting to form the compound according to formula V is performed in the presence of tetraalkylammonium ions, iodide ions, or a mixture thereof. In some embodiments, the reacting to form the compound according to formula V is performed in the presence of tetraalkylammonium ions and iodide ions. In some embodiments, the tetraalkylammonium ions are tetra-n-butylammonium ions.

In some embodiments, the reacting to form the compound according to formula V may be performed at ambient temperature, or may be performed at an elevated temperature. In some embodiments, the reacting to form the compound according to formula V is performed at a temperature in the range from about 30° C. to about 120° C. In some embodiments, the reacting to form the compound according to formula V is performed at a temperature in the range from about 50° C. to about 100° C. In some embodiments, the reacting to form the compound according to formula V is performed at a temperature in the range from about 60° C. to about 80° C.

When the process is performed using the compound according to formula VI wherein R¹ is hydrogen, or a salt of such a compound, the carboxylate group may be alkylated in addition to the sulfinate group, and thereby form an ester. Alternatively, the moiety —C(═O)OR¹ of the compound according to formula VI used as a starting material for the process may be an ester. In either case, the product of the reaction of the compound according to formula VI with the compound according to formula VII may comprise a compound that is in the form of a carboxylate ester. If it is desired to obtain a compound according to formula V in the form of an acid, the carboxylate ester may be hydrolyzed to form a compound according formula V that is in the form of an acid.

Accordingly, some embodiments of the process for preparing the compound according to formula V further comprising hydrolyzing a carboxylate ester co-product from the reacting of the compound according to formula VI with the compound according to formula VII, by treatment with a hydrolyzing base under conditions sufficient to hydrolyze the ester group of the co-product. Examples of conditions that may be used to hydrolyze an ester include using a strong base in a water-containing solvent medium, or using a metal hydroxide as the base (e.g. an alkali metal hydroxide, which may be used in water or a hydroxylic solvent such as methanol). An example of a suitable base is an alkali metal base such as sodium or potassium hydroxide. In some embodiments, the hydrolyzing base comprises sodium hydroxide.

The compound according to formula VII may be used in excess relative to the amount of the compound according to formula VI. Due to the competing alkylation of a carboxyl group when R¹ is hydrogen in the compound according to formula VI, it may be desirable to use at least about two equivalents of the compound according to formula VII, for example about three or more equivalents, or about four or more equivalents. In an example of an embodiment of the process, about four equivalents may be used. When a tetraalkylammonium salt, for example a tetra-n-butylammonium salt, or an iodide salt is used, then the amount used may be a catalytic amount, i.e. less than about one equivalents, such as about 0.1 equivalents. In an example of an embodiment of the process, about 0.1 equivalents of tetra-n-butylammonium iodide is used as a catalyst.

The process described herein for the preparation of a compound according to formula V, or any of the embodiments thereof, may optionally be used for the synthesis of the compound according to formula V to be used in the aforementioned process for the synthesis of the compound according to formula IV, or any of the embodiments of such a process, and which may further be used in the aforementioned process for the synthesis of the compound according to formula II, or a salt thereof, or any of the embodiments of such a process, and which may yet further be used in the aforementioned process for the synthesis of the compound according to formula I, or a salt thereof, or any of the embodiments of such a process.

### 5. Preparation of the Compound According to Formula VI by Reducing a Sulfonyl Chloride According to Formula VIII (Step 2)

In another aspect there is provided a process for preparing a compound according to formula VI:

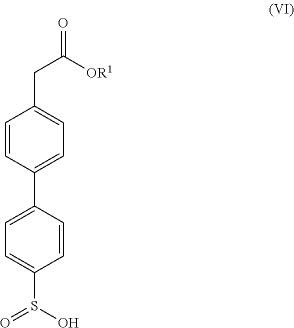

or a salt thereof, wherein R¹ is hydrogen or C₁-C₆ alkyl, comprising reducing a compound according to formula VIII:

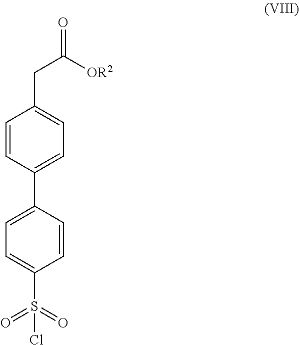

or a salt thereof, wherein R² is hydrogen or C₁-C₆ alkyl.

In some embodiments, R² is hydrogen.

In some embodiments, reducing the compound according to formula VIII or salt thereof is performed in the presence of a suitable a reducing agent. Suitable reducing agents include metal sulfite salts, for example sodium sulfite. Other suitable reducing agents include sulfite or bisulfites, specifically, for example, sodium sulfite, potassium sulfite, sodium bisulfite, and potassium bisulfite. The amount of the reducing agent typically used is usually an excess relative to the amount of the sulfonyl chloride, for example an amount in the range of about 1 to about 4 equivalents, for example about 3 equivalents. In some embodiments, the reducing agent for reducing the compound according to formula VIII or salt thereof comprises a metal sulfite salt. In some embodiments, the metal sulfite salt is sodium sulfite.

In some embodiments, the reduction of the compound according to formula VIII or salt thereof is performed in a solution comprising water.

The reducing of the compound according to formula VIII or salt thereof is typically carried out in the presence of a base. Suitable bases include alkali metal hydroxides, alkali metal carbonates, alkali metal bicarbonate, alkali metal phosphates and the like. Examples of suitable bases include sodium hydroxide, potassium hydroxide, sodium carbonate, potassium carbonate, sodium bicarbonate, potassium bicarbonate, and the like. Usually, the amount of base used is within the range of about 1 to 4 equivalents.

In some embodiments, the reducing of the compound according to formula VIII or salt thereof may be performed at about ambient temperature or higher. In some embodiments, the reducing of the compound according to formula VIII or salt thereof is performed at a temperature in the range from about 40° C. to about 100° C. In some embodiments, the reducing of the compound according to formula VIII or salt thereof is performed at a temperature in the range from about 40° C. to about 80° C. In some embodiments, the reducing of the compound according to formula VIII or salt thereof is performed at a temperature in the range from about 50° C. to about 70° C.

The process described herein for the preparation of a compound according to formula VI, or any of the embodiments thereof, may optionally be used for the synthesis of the compound according to formula VI to be used in the aforementioned process for the synthesis of the compound according to formula V, or any of the embodiments of such a process, and which may further be used in the aforementioned process for the synthesis of the compound according to formula IV, or any of the embodiments of such a process, which may further be used in the aforementioned process for the synthesis of the compound according to formula II, or a salt thereof, or any of the embodiments of such a process, and which may yet further be used in the aforementioned process for the synthesis of the compound according to formula I, or a salt thereof, or any of the embodiments of such a process.

### 6. Preparation of the Compound According to Formula VIII by Chlorosulfonation of a 4-Biphenylacetic Acid Derivative According to Formula IX (Step 1)

A process is also provided for preparing a compound according to formula VIII:

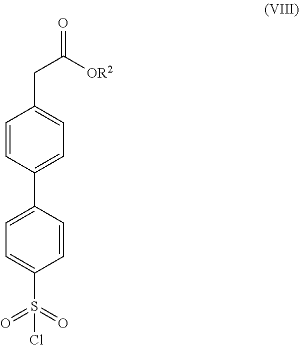

or a salt thereof, wherein R² is hydrogen or C₁-C₆ alkyl, comprising chlorosulfonating a compound according to formula IX:

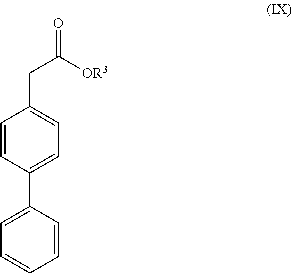

or a salt thereof, wherein R³ is hydrogen or C₁-C₆ alkyl.

In some embodiments, R³ is hydrogen.

In some embodiments, the chlorosulfonating is performed in the presence of a suitable chlorosulfonating agent. In some embodiments, the chlorosulfonating agent is chlorosulfonic acid. The amount used of the chlorosulfonating agent such as chlorosulfonic acid may be an excess, for example an amount in the range from about one to about 10 equivalents. When chlorosulfonic acid is used, the use of an excess of the reagent is not considered to be detrimental because the its hydrolysis products are water soluble and readily separated from the product. For example, a suitable amount may be in the range from about two to about 10 equivalents, for example about seven equivalents.

In general, the chlorosulfonating may be performed in any solvent in which the compound according to formula IX at least partially dissolves and which does not react with the chlorosulfonic acid, for example chlorinated hydrocarbons or carboxylic acid solvents. In some embodiments, the chlorosulfonating is performed in a carboxylic acid solvent. In some embodiments, the carboxylic acid solvent is trifluoroacetic acid.

The chlorosulfonating is typically performed at a temperature with cooling. In some embodiments, the chlorosulfonating is performed at a temperature in the range from about 0° C. to about 40° C. In some embodiments, the chlorosulfonating is performed at a temperature in the range from about 10° C. to about 30° C. In some embodiments, the chlorosulfonating is performed at a temperature in the range from about 20° C. to about 30° C.

The process described herein for the preparation of a compound according to formula VIII, or any of the embodiments thereof, may optionally be used for the synthesis of the compound according to formula VIII to be used in the aforementioned process for the synthesis of the compound according to formula VI, or any of the embodiments thereof, which may optionally be used in the aforementioned process for the synthesis of the compound according to formula V, or any of the embodiments of such a process, and which may further be used in the aforementioned process for the synthesis of the compound according to formula N, or any of the embodiments of such a process, which may further be used in the aforementioned process for the synthesis of the compound according to formula II, or a salt thereof, or any of the embodiments of such a process, and which may yet further be used in the aforementioned process for the synthesis of the compound according to formula I, or a salt thereof, or any of the embodiments of such a process.

### 7. Processes for Preparing Salts of the Compound According to Formula I.

In another aspect, there is provided a process for preparing a salt of a compound according to formula I, comprising reacting the compound according to formula I with an acid and isolating a salt of the compound according to formula I.

The term “salts” used in reference to the compound of formula I embraces any acid addition salts. The term “pharmaceutically-acceptable salt” refers to salts that possess toxicity profiles within a range that affords utility in pharmaceutical applications. Pharmaceutically unacceptable salts may nonetheless possess properties such as high crystallinity, which may render them useful. The person skilled in the art will know how to prepare and select suitable pharmaceutically acceptable salt forms for example, as described in Handbook of Pharmaceutical Salts: Properties, Selection, and Use by P. H. Stahl and C. G. Wermuth (Wiley-VCH 2002).

In some embodiments of the process for making a salt of the compound according to formula I, the acid that is reacted with the compound according to formula I is citric acid and the salt is a citrate. In some embodiments, the salt is a citrate. In some embodiments thereof, the salt is a mono-citrate (i.e. a salt comprising the compound according to formula I and citric acid in a molar ratio of about 1:1). In other embodiments, the salt is a di-citrate (i.e. a salt comprising the compound according to formula I and citric acid in a molar ratio of about 1:2). In some embodiments of the process for making a salt of the compound according to formula I, the acid that is reacted with the compound according to formula I is hydrochloric acid and the salt is a hydrochloride salt. In some embodiments of the process for making a salt of the compound according to formula I, the acid that is reacted with the compound according to formula I is maleic acid and the salt is a maleate salt.

For acids in which it is possible to form different salts which vary in the relative molar ratio of the compound according to formula I and the acid present in the salt, the salt which is prepared may be determined by controlling the relative molar amounts of the compound according to formula I and the acid which are used in the process for forming the salt. For example in order to form the mono-citrate salt of the compound according to formula I a molar ratio of citric acid relative to the compound according to formula I of about 1:1 may be used. On the other hand, in order to form the di-citrate salt, a molar ratio of citric acid relative to the compound of formula I of about 2:1 may be used. In such cases where salts having differing stoichiometries may form, it is desirable to select the reaction conditions for the reaction of the compound according to formula I with the acid such that a homogenous mixture comprising the requisite amounts of the compound according to formula I and the acid in a solvent is formed prior to commencement of crystallization of the salt from the reaction mixture.

In some embodiments of the process for forming a citrate salt, for example a mono-citrate or a di-citrate salt, the solvent used for reacting the compound according to formula I and citric acid to form the salt is (or comprises) a C₂-C₄ alkanonitrile such as acetonitrile. In other embodiments, the salt is formed in a solvent other than acetonitrile or solvent mixtures comprising acetonitrile.

A process is provided for preparing a citrate salt of a compound according to formula I comprising reacting a compound according to formula I with citric acid in a solvent. In some embodiments, the solvent is, or comprises, acetonitrile. In other embodiments, the solvent is other than acetonitrile. In other embodiments the solvent is other than a solvent mixture comprising acetonitrile. In some embodiments thereof, the salt is a mono-citrate. In other embodiments thereof, the salt is a di-citrate. In some embodiments, the solvent comprises a C₃-C₅ alkanone. In some embodiments, the C₃-C₅ alkanone is 2-butanone. In some embodiments, the solvent further comprises a C₁-C₄ alkanol. In some embodiments, the C₁-C₄ alkanol is methanol. In some embodiments of such a process, the compound according to formula I is dissolved in an organic solvent such as a C₃-C₅ alkanone, for example 2-butanone, and reacted with citric acid dissolved in water or a suitable polar solvent such as a C₁-C₄ alkanol, for example methanol. The mixture may be initially formed (or warmed to) a temperature sufficient to form a homogenous mixture comprising the compound according to formula I and the citric acid, from which the salt crystallizes upon cooling and/or addition of a less polar solvent. For example, in a particular embodiment, about the compound according to formula I in 2-butanone and about 2 equivalents of citric acid in methanol are combined and heated to temperature in the range from about 50° C. to about 70° C. (for example about 60° C.), and then cooled to a temperature from about 0° C. to about 10° C., and the resulting precipitated solid of the di-citrate salt is collected by filtration.

The compound (R)-1-{2-[4′-(3-methoxy-propane-1-sulfonyl)-biphenyl-4-yl]-ethyl}-2-methyl-pyrrolidine di-citrate is described in PCT Application PCT/US2008/07144, which is incorporated herein by reference in its entirety.

In some embodiments of the aforementioned processes for forming a salt of the compound according to formula I, the compound according to formula I is prepared according to one of the aforementioned methods for synthesizing the compound according to formula I. There are thus provided methods for synthesizing a salt of the compound according to formula I comprising any of the aforementioned methods for preparing the compounds according to formula I, which further comprise any of the processes described herein for forming the salt of the compound according to formula I by reacting the compound according to formula I with a suitable acid.

### III. Intermediates

Also provided as an aspect of the invention are intermediates that are useful in the synthesis of the compound according to formula I, and salts thereof.

As one aspect there is provided a compound according to formula XI:

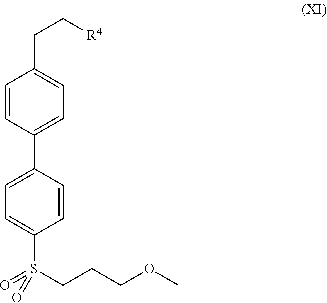

wherein R⁴ is chloride, bromide, iodide, hydroxyl, or a sulfonate ester.

In some embodiments R⁴ is iodide, hydroxyl, or a sulfonate ester

In some embodiments thereof, R⁴ is hydroxyl.

In some embodiments thereof, R⁴ is a sulfonate ester, for example a methanesulfonate ester.

Also provided is a compound according to formula V:

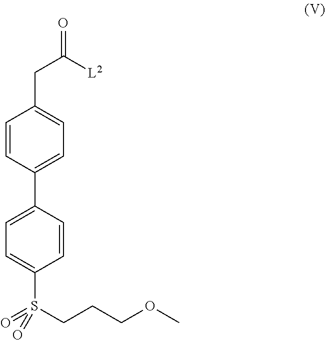

wherein L² is hydroxyl, or a salt of the hydroxyl, or L² is C₁-C₆ alkoxy.

In some embodiments thereof, L² is hydroxyl.

Also provided is a compound according to formula VI:

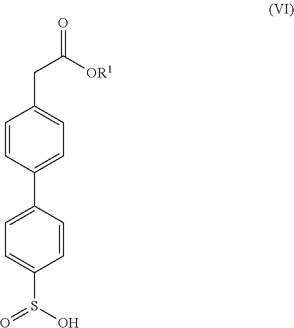

or a salt thereof, wherein R¹ is hydrogen or C₁-C₆ alkyl.

In some embodiments thereof, R¹ is hydrogen.

Also provided is a compound according to formula VIII:

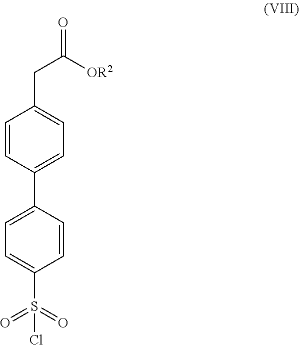

or a salt thereof, wherein R² is hydrogen or C₁-C₆ alkyl.

In embodiments thereof, R² is hydrogen.

### IV. Manufacture of Pharmaceutical Products

Following synthesis by the methods described herein, the compound according to Formula I, or the salt thereof, such as the mono-citrate or di-citrate, may be used for the manufacture of pharmaceutical products. In turn, the pharmaceutical products may be useful for the treatment of various diseases and conditions for which histamine H3-receptor modulators are indicated.

Pharmaceutical compositions may be prepared by any suitable method, typically by uniformly mixing the active compound(s) with liquids or finely divided solid carriers, or both, in the required proportions, and then, if necessary, forming the resulting mixture into a desired shape.

Accordingly, there are provided processes for preparing pharmaceutical compositions comprising admixing (R)-1-{2-[4′-(3-methoxy-propane-1-sulfonyl)-biphenyl-4-yl]-ethyl}-2-methyl-pyrrolidine or any salt thereof, such as a mono- or di-citrate, prepared by any of the methods described herein, and a pharmaceutically acceptable carrier.

Conventional excipients, such as binding agents, fillers, acceptable wetting agents, tableting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of a dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound according to formula I can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Ed., 2000, Lippincott Williams & Wilkins, (Editors: Gennaro, A. R., et al.).

While it is possible that a compound or salt thereof as described herein may, in an alternative use, be administered as a raw or pure chemical, it is preferable however to present the compound or active ingredient as a pharmaceutical formulation or composition further comprising a pharmaceutically acceptable carrier. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient may also be administered by injection as a composition wherein, for example, saline, dextrose or water may be used as a suitable pharmaceutically acceptable carrier.

The dose when using the compounds of the present invention can vary within wide limits, as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg, and about 0.001 mg to about 25 mg. Multiple doses may be administered during the day, especially when relatively large amounts are deemed to be needed, for example 2, 3 or 4, doses. Depending on the individual and as deemed appropriate from the patient's physician or caregiver it may be necessary to deviate upward or downward from the doses described herein.

The amount of active ingredient, required for use in treatment will vary with not only the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, whether the disease state is chronic or acute, whether treatment or prophylaxis is conducted, or on whether further active compounds are administered in addition to the compounds of the present invention and as part of a drug combination. The dosage regimen for treating a disease condition with the compounds and/or compositions of this invention is selected in accordance with a variety of factors as cited above. Thus, the actual dosage regimen employed may vary widely and therefore may deviate from a preferred dosage regimen and one skilled in the art will recognize that dosages and dosage regimens outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds and crystalline forms thereof, according to the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may contain from 0.5 to about 90 percent of the active compound; however, an artisan of ordinary skill would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as solvents or suspending media. For this purpose, any bland fixed oil may be employed including synthetic mono- or diglycerides. In addition, fatty acids such as oleic acid find use in the preparation of injectables.

The compounds and crystalline forms thereof, according to the present invention, may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis, the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will generally also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example, carbon dioxide, CFCs, such as, dichlorodifluoromethane, trichlorofluoromethane, and dichlorotetrafluoroethane, HFAs, such as, 1,1,1,2,3,3,3-heptaflurorpropane and 1,1,1,2-tetrafluoroethane, and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively, the active ingredients may be provided in the form of a dry powder, for example, a powder mix of the compound in a suitable powder base such as lactose, starch, starch derivatives such as hydroxypropylmethylcellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

Some embodiments of the present invention include a method of producing a pharmaceutical composition for “combination-therapy” comprising admixing at least one compound or crystalline form thereof as disclosed herein, together with at least one known pharmaceutical agent as described herein and a pharmaceutically acceptable carrier.

It is noted that when the H3-receptor modulators are utilized as active ingredients in a pharmaceutical composition, these are not intended for use only in humans, but in other non-human mammals as well. Indeed, recent advances in the area of animal health-care suggest that consideration be given for the use of active agents, such as H3-receptor modulators, for the treatment of an H3-receptor associated disease or disorder in companionship animals (e.g., cats, dogs, etc.) and in livestock animals (e.g., cows, chickens, fish, etc.) Those of ordinary skill in the art are readily credited with understanding the utility of such compounds in such settings.

The formulations prepared by the methods described herein are useful for the synthesis of any disease or condition for which the administration of a histamine H3 receptor modulator is indicated.

Histamine [2-(imidazol-4-yl)ethylamine] exerts its physiological effects through four distinct G-protein coupled receptors (GPCRs), termed H1, H2, H3 and H4. The histamine H3-receptor was first identified in 1983, when it was determined that the H3-receptor acted as an autoreceptor controlling both the synthesis and release of histamine (see: Arrang et al. Nature 1983, 302, 832-7). At least four human and three rat splice variants have proven functional activity in pharmacological assays (Passani et al., Trends in Pharmacol. Sci. 2004, 25, 618-625). Rat and human histamine H3-receptors also show constitutive activity which means that they can transduce a signal even in the absence of a ligand. Histamine H3-receptors also function as heteroceptors, modulating the release of a number of other transmitter substances including serotonin, acetylcholine, dopamine and noradrenaline (see: Brown et al. Prog. Neurobiol. 2001, 63, 637-672). Thus, there are a number of therapeutic applications for ligands that target the histamine H3-receptor, where the ligand functions as either an antagonist or inverse agonist (for reviews, see: Leurs et al., Nat. Rev. Drug. Discov., 2005, 4, 107-120; Passani et al., Trends Pharmacol. Sci. 2004, 25, 618-625).

Accordingly, preclinical studies have identified a number of indications which are amenable to treatment with histamine H3-receptor antagonists and inverse agonists, such as compounds of the present invention. The compounds disclosed herein are believed to be useful in the treatment and/or prevention of several diseases and disorders, and in the amelioration of symptoms thereof. These compounds can be used alone or in combination with other compounds for the treatment and/or prevention of diseases and disorders. Without limitation, these diseases and disorders include the following.

Histamine H3-receptor antagonists have been shown to increase wakefulness (e.g. Lin J. S. et al. Brain Research 1990, 523, 325-330). This effect demonstrates that H3-receptor antagonists can be useful for treating disorders of sleep and wakefulness (Parmentier et al. J Neurosci. 2002, 22, 7695-7711; Ligneau et al. J. Pharmacol. Exp. Ther. 1998, 287, 658-666). For example, histamine H3-receptor antagonists and inverse agonists can be used to treat the somnolence syndrome associated with different pathological conditions, such as, sleep apnea and Parkinson's disease or circumstances associated with lifestyle, such as, daytime somnolence from sleep deprivation as a result of nocturnal jobs, overwork, or jet-lag (see Passani et al., Trends Pharmacol. Sci., 2004, 25, 618-625). Somnolence is a major public health problem because of its high prevalence (19-37% of the general population) and risk for causing work and traffic accidents.

Sleep apnea (alternatively sleep apnoea) is a common sleep disorder characterized by brief interruptions of breathing during sleep. These episodes, called apneas, last 10 seconds or more and occur repeatedly throughout the night. People with sleep apnea partially awaken as they struggle to breathe, but in the morning they may not be aware of the disturbances in their sleep. The most common type of sleep apnea is obstructive sleep apnea (OSA), caused by relaxation of soft tissue in the back of the throat that blocks the passage of air. Central sleep apnea (CSA) is caused by irregularities in the brain's normal signals to breathe. The hallmark symptom of the disorder is excessive daytime sleepiness. Additional symptoms of sleep apnea include restless sleep, loud snoring (with periods of silence followed by gasps), falling asleep during the day, morning headaches, trouble concentrating, irritability, forgetfulness, mood or behavior changes, weight gain, increased heart rate, anxiety, and depression.

Few drug-based treatments of obstructive sleep apnea are known despite over two decades of research and tests. Oral administration of the methylxanthine theophylline (chemically similar to caffeine) can reduce the number of episodes of apnea, but can also produce side effects such as palpitations and insomnia. Theophylline is generally ineffective in adults with OSA, but is sometimes used to treat CSA, and infants and children with apnea. In 2003 and 2004, some neuroactive drugs, particularly modern-generation antidepressants including mirtazapine, have been reported to reduce incidences of obstructive sleep apnea. When other treatments do not completely treat the OSA, drugs are sometimes prescribed to treat a patient's daytime sleepiness or somnolence. These range from stimulants such as amphetamines to modern anti-narcoleptic medicines. The drug modafinil is seeing increased use in this role as of 2004.

In addition, for example, histamine H3-receptor antagonists and inverse agonists can be used to treat narcolepsy (Tedford et al. Soc. Neurosci. Abstr. 1999, 25, 460.3). Narcolepsy is a neurological condition most often characterized by Excessive Daytime Sleepiness (EDS), episodes of sleep and disorder of REM or rapid eye movement sleep. The main characteristic of narcolepsy is overwhelming Excessive Daytime Sleepiness (EDS), even after adequate night-time sleep. A person with narcolepsy is likely to become drowsy or to fall asleep, often at inappropriate times and places. In addition, night-time sleep may be fragmented with frequent awakenings. Classic symptoms of narcolepsy include, for example, cataplexy which is sudden episodes of loss of muscle function, ranging from slight weakness (such as limpness at the neck or knees, sagging facial muscles, or inability to speak clearly) to complete body collapse. Episodes may be triggered by sudden emotional reactions such as laughter, anger, surprise, or fear, and may last from a few seconds to several minutes. Another symptom of narcolepsy is sleep paralysis, which is the temporary inability to talk or move when waking up. Other symptoms include, for example, hypnagogic hallucinations which are vivid, often frightening, dream-like experiences that occur while dozing, falling asleep and/or while awakening, and automatic behavior which occurs when a person continues to function (talking, putting things away, etc.) during sleep episodes, but awakens with no memory of performing such activities. Daytime sleepiness, sleep paralysis, and hypnagogic hallucinations also occur in people who do not have narcolepsy, such as in people who are suffering from extreme lack of sleep. Cataplexy is generally considered unique to narcolepsy.

Currently the treatments available for narcolepsy treat the symptoms, but not the underlying cause. For cataplexy and REM-sleep symptoms, antidepressant medications and other drugs that suppress REM sleep are prescribed. The drowsiness is normally treated using stimulants such as methylphenidate (Ritalin), amphetamines (Adderall), dextroamphetamine (Dexedrine), methamphetamine (Desoxyn), modafinil (Provigil), etc. Other medications used are codeine and selegiline. The cataplexy is treated using clomipramine, imipramine, or protriptyline but this need only be done in severe cases. The drug gamma-hydroxybutyrate (GHB) (Xyrem) is approved in the USA by the Food and Drug Administration to treat both the cataplexy and excessive daytime sleepiness associated with narcolepsy.

Interestingly, modafinil (Provigil) has recently been shown to increase hypothalamic histamine release (Ishizuka et al. Neurosci. Lett. 2003, 339, 143-146).

In addition, recent studies using the classic Doberman model of narcolepsy with a non-imidazole histamine H3-receptor antagonist showed that a histamine H3-receptor antagonist can reduce the number of cataplectic attacks and the duration of the attacks (Carruthers Ann. Meet. Eur. Histamine Res. Soc. 2004, Abs. p 31).

In summary, histamine H3-receptor antagonists and inverse agonists can be used for the treatment and/or prevention of conditions associated with excessive daytime sleepiness such as hypersomnia, narcolepsy, sleep apnea, time zone change disorder, and other disorders which are associated with excessive daytime sleepiness such as fibromyalgia, and multiple sclerosis (Parmentier et al., J. Neurosci. 2002, 22, 7695-7711; Ligneau et al. J. Pharmacol. Exp. Ther. 1998, 287, 658-666). Other conditions include excessive sleepiness due to shift work, medical disorders, psychiatric disorders, narcolepsy, primary hypersomnia, and the like. Histamine H3-receptor antagonists and inverse agonists can also be used occasionally to promote wakefulness or vigilance in shift workers, sleep deprivation, post anaesthesia grogginess, drowsiness as a side effect from a medication, military use and the like.

In addition, wakefulness is a prerequisite for several brain functions including attention, learning, and memory and is required for appropriate behaviors in response to environmental challenges. Histamine H3-receptor antagonists and inverse agonists have been shown to improve cognitive performance in various animal models (Hancock and Fox in Milestones in Drug Therapy, ed. Buccafusco, 2003). These compounds can be used as pro-cognitive agents and can increase vigilance. Therefore, histamine H3-receptor antagonists and inverse agonists can be used in aging or degenerative disorders in which vigilance, attention and memory are impaired, for example, as in Alzheimer's disease or other dementias.

Alzheimer's disease (AD), a neurodegenerative disorder, is the most common cause of dementia. It is characterized clinically by progressive cognitive deterioration together with neuropsychiatric symptoms and behavioral changes. The most striking early symptom is memory loss, which usually manifests as minor forgetfulness that becomes steadily more pronounced with illness progression, with relative preservation of older memories. As the disorder progresses, cognitive (intellectual) impairment extends to the domains of language, skilled movements, recognition and functions closely related to the frontal and temporal lobes of the brain such as decision-making and planning. There is currently no cure for AD, although there are drugs which offer symptomatic benefit, specifically with respect to short-term memory impairment. These drugs include acetylcholinesterase inhibitors such as donepezil (Aricept), galantamine (Razadyne) and rivastigmine (Exelon) and NMDA antagonists such as memantine.

Histamine H3-receptor antagonists and inverse agonists can be used to treat or prevent cognitive disorders (Passani et al. Trends Pharmacol. Sci. 2004, 25, 618-625), epilepsy (Vohora et al. Pharmacol. Biochem. Behav. 2001, 68, 735-741), depression (Perez-Garcia et al. Psychopharmacol. 1999, 142, 215-220), attention deficit hyperactivity disorder (ADHD), (Fox et al. Behav. Brain Res. 2002, 131, 151-61), and schizophrenia (Fox et al. J. Pharmacol. Exp. Ther. 2005, 313, 176-190). These indications are described briefly below. For additional information, see reviews by Leurs et al., Nat. Rev. Drug. Discov. 2005, 4, 107-120, and Vohora Investigational Drugs 2004, 7, 667-673). Histamine H3-receptor antagonists or inverse agonists can also be used as a novel therapeutic approach to restore cortical activation in comatose or brain-traumatized patients (Passani et al., Trends in Pharmacol. Sci. 2004, 25, 618-625).

As stated above, histamine H3-receptor antagonists and inverse agonists can be used to treat or prevent epilepsy. Epilepsy (often referred to as a seizure disorder) is a chronic neurological condition characterized by recurrent unprovoked seizures. In terms of their pattern of activity, seizures may be described as either partial (focal) or generalized. Partial seizures only involve a localized part of the brain, whereas generalized seizures involve the entire cortex. There are many different epilepsy syndromes, each presenting with its own unique combination of seizure type, typical age of onset, EEG findings, treatment, and prognosis. Some common seizure syndromes include, for example, infantile spasms (West syndrome), childhood absence epilepsy, and benign focal epilepsy of childhood (Benign Rolandic epilepsy), juvenile myoclonic epilepsy, temporal lobe epilepsy, frontal lobe epilepsy and Lennox-Gastaut syndrome.

Compounds of the present invention can be used in combination with various known drugs. For example, compounds of the present invention can be used with one or more drugs that prevent seizures or reduce seizure frequency: these include carbamazepine (common brand name Tegretol), clobazam (Frisium), clonazepam (Klonopin), ethosuximide (Zarontin), felbamate (Felbatol), fosphenytoin (Cerebyx), flurazepam (Dalmane), gabapentin (Neurontin), lamotrigine (Lamictal), levetiracetam (Keppra), oxcarbazepine (Trileptal), mephenytoin (Mesantoin), phenobarbital (Luminal), phenytoin (Dilantin), pregabalin (Lyrica), primidone (Mysoline), sodium valproate (Epilim), tiagabine (Gabitril), topiramate (Topamax), valproate semisodium (Depakote), valproic acid (Depakene, Convulex), and vigabatrin (Sabril). Other drugs are commonly used to abort an active seizure or interrupt a seizure flurry; these include diazepam (Valium) and lorazepam (Ativan). Drugs used only in the treatment of refractory status epilepticus include paraldehyde (Paral) and pentobarbital (Nembutal).

As stated above, a histamine H3-receptor antagonist or inverse agonist can be used as the sole agent of treatment or can be used in combination with other agents. For example, Vohora et al. show that a histamine H3-receptor antagonist can work as an anti-epilepsy, anti-seizure drug and also showed effect with sub-effective doses of the H3-receptor antagonist in combination with sub-effective doses of known anti-epileptic drugs (Vohora et al. Pharmacol. Biochem. Behav. 2001, 68, 735-741).

Perez-Garcia et al. (Psychopharmacol. 1999, 142, 215-220) tested the ability of a histamine H3-receptor agonist and antagonist on experimental mouse models of anxiety (elevated plus-maze) and depression (forced swimming test). They found that while the compounds did not have a significant effect on the model of anxiety, a H3-receptor antagonist did have a significant dose-dependent effect in the model of depression. Thus, histamine H3-receptor antagonists or inverse agonists can have antidepressant effects.

Clinical depression is a state of sadness or melancholia that has advanced to the point of being disruptive to an individual's social functioning and/or activities of daily living. Clinical depression affects about 16% of the population on at least one occasion in their lives. Clinical depression is currently the leading cause of disability in the U.S. as well as other countries, and is expected to become the second leading cause of disability worldwide (after heart disease) by the year 2020, according to the World Health Organization.

Compounds of the present invention can be used in combination with various known drugs. For examples, compounds of the present invention can be used with one or more of the drugs currently available that can relieve the symptoms of depression. They include, for example, monoamine oxidase inhibitors (MAOIs) such as Nardil or Moclobemide (Manerix), tricyclic antidepressants, selective serotonin reuptake inhibitors (SSRIs) such as fluoxetine (Prozac), paroxetine (Paxil), escitalopram (Lexapro), and sertraline (Zoloft), norepinephrine reuptake inhibitors such as reboxetine (Edronax), and serotonin-norepinephrine reuptake inhibitors (SNRIs) such as venlafaxine (Effexor) and duloxetine (Cymbalta).

As stated above, histamine H3-receptor antagonists and inverse agonists can be used to treat or prevent attention deficit hyperactivity disorder (ADHD). According to the Diagnostic and Statistical Manual of Mental Disorders-IV-TR, ADHD is a developmental disorder that arises in childhood, in most cases before the age of 7 years, is characterized by developmentally inappropriate levels of inattention and/or hyperactive-impulsive behavior, and results in impairment in one or more major life activities, such as family, peer, educational, occupational, social, or adaptive functioning. ADHD can also be diagnosed in adulthood.

The first-line medications used to treat ADHD are mostly stimulants, which work by stimulating the areas of the brain responsible for focus, attention, and impulse control. The use of stimulants to treat a syndrome often characterized by hyperactivity is sometimes referred to as a paradoxical effect, but there is no real paradox in that stimulants activate brain inhibitory and self-organizing mechanisms permitting the individual to have greater self-regulation. The stimulants used include, for example, methylphenidate (sold as Ritalin, Ritalin SR and Ritalin LA), Metadate, Metadate ER, Metadate CD, Concerta, Focalin, Focalin XR or Methylin. The stimulants also include, for example, amphetamines such dextroamphetamine, sold as Dexedrine, Dexedrine Spansules, Adderall, and Adderall XR, a trade name for a mixture of dextroamphetamine and laevoamphetamine salts, methamphetamine sold as Desoxyn, bupropion, a dopamine and norepinephrine reuptake inhibitor, marketed under the brand name Wellbutrin. A non-stimulant medication to treat ADHD is Atomoxetine (sold as Strattera) a norepinephrine reuptake inhibitor. Other drugs sometimes used for ADHD include, for example, benzphetamine (Didrex), Provigil/Alertec/modafinil and clonidine. Recently it has been reported that in a rat pup model for ADHD, a histamine H3-receptor antagonist was at least as effective as methylphenidate (Ritalin) (Hancock and Fox in Milestones in Drug Therapy, ed. Buccafusco, 2003). Compounds of the present invention can be used in combination with various known drugs. For examples, compounds of the present invention can be used with one or more of the drugs used to treat ADHD and related disorders.

As stated above, histamine H3-receptor antagonists and inverse agonists can be used to treat or prevent schizophrenia. Schizophrenia is a psychiatric diagnosis that describes a mental disorder characterized by impairments in the perception or expression of reality and by significant social or occupational dysfunction. A person experiencing untreated schizophrenia is typically characterized as demonstrating disorganized thinking, and as experiencing delusions or auditory hallucinations. Although the disorder is primarily thought to affect cognition, it can also contribute to chronic problems with behavior and emotion. Schizophrenia is often described in terms of “positive” and “negative” symptoms. Positive symptoms include delusions, auditory hallucinations and thought disorder, and are typically regarded as manifestations of psychosis. Negative symptoms are so named because they are considered to be the loss or absence of normal traits or abilities, and include features such as flat, blunted or constricted affect and emotion, poverty of speech and lack of motivation. Some models of schizophrenia include formal thought disorder and planning difficulties in a third group, a “disorganization syndrome.”

The first line pharmacological therapy for schizophrenia is usually the use of antipsychotic medication. Antipsychotic drugs are only thought to provide symptomatic relief from the positive symptoms of psychosis. The newer atypical antipsychotic medications (such as clozapine, risperidone, olanzapine, quetiapine, ziprasidone and aripiprazole) are usually preferred over older typical antipsychotic medications (such as chlorpromazine and haloperidol) due to their favorable side-effect profile. While the atypical antipsychotics are associated with less extra pyramidal side effects and tardive dyskinesia than the conventional antipsychotics, some of the agents in this class (especially olanzapine and clozapine) appear to be associated with metabolic side effects such as weight gain, hyperglycemia and hypertriglyceridemia that must be considered when choosing appropriate pharmacotherapy.

Histamine H3-receptor antagonists or inverse agonists can be used to treat obesity (Hancock, Curr. Opin. Investig. Drugs 2003, 4, 1190-1197). The role of neuronal histamine in food intake has been established for many years and neuronal histamine release and/or signaling has been implicated in the anorectic actions of known mediators in the feeding cycle such as leptin, amylin and bombesin. In the brain, the H3-receptor is implicated in the regulation of histamine release in the hypothalamus. Moreover, in situ hybridization studies have revealed histamine H3-receptor mRNA expression in rat brown adipose tissue, indicating a role in the regulation of thermogenesis (Karlstedt et al., Mol. Cell. Neurosci. 2003, 24, 614-622). Furthermore, histamine H3-receptor antagonists have been investigated in various preclinical models of obesity and have shown to be effective in reducing food intake, reducing weight, and decreasing total body fat in mice (Hancock, et al. Eur. J. Pharmacol. 2004, 487, 183-197). The most common drugs used for the treatment of obesity are sibutramine (Meridia) and orlistat (Xenical), both of which have limited effectiveness and significant side effects. Therefore, novel anti-obesity agents, such as histamine H3-receptor antagonists or inverse agonists, are needed.

Histamine H3-receptor antagonists or inverse agonists can also be used to treat upper airway allergic responses (U.S. Pat. Nos. 5,217,986; 5,352,707 and 5,869,479) including allergic rhinitis and nasal congestion. Allergic rhinitis is a frequently occurring chronic disease that affects a large number of people. Recent analysis of histamine H3-receptor expression in the periphery by quantitative PCR revealed that H3-receptor mRNA is abundantly expressed in human nasal mucosa (Varty et al. Eur. J. Pharmacol. 2004, 484, 83-89). In addition, in a cat model of nasal decongestion, a combination of histamine H3-receptor antagonists with the H1 receptor antagonist chlorpheniramine resulted in significant nasal decongestion without the hypertensive effect seen with adrenergic agonists. (McLeod et al. Am. J. Rhinol. 1999, 13, 391-399). Thus, histamine H3-receptor antagonists or inverse agonists can be used alone or in combination with H1 receptor blockage for the treatment of allergic rhinitis and nasal congestion.

Histamine H3-receptor antagonists or inverse agonists have therapeutic potential for the treatment of pain (Medhurst et al. Biochemical Pharmacology (2007), 73(8), 1182-1194).

The compound (R)-1-{2-[4′-(3-methoxy-propane-1-sulfonyl)-biphenyl-4-yl]-ethyl}-2-methyl-pyrrolidine and salts thereof, have activity as histamine H3-receptor modulators. Accordingly, such compounds prepared by the methods described herein can be used in methods of modulating the histamine H3-receptor by contacting the receptor, and hence in methods of treatment (as described herein) wherein such biological activity exerts a useful effect.

### Examples

The following non-limiting examples are provided to illustrate the invention.

### Example 1

### Synthesis of 2-(4′-(Chlorosulfonyl)biphenyl-4-yl)acetic Acid by Chlorosulfonation of 2-(Biphenyl-4-yl)acetic Acid

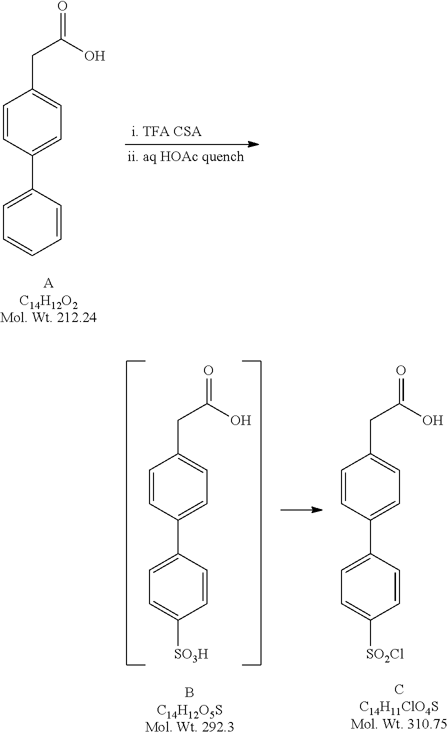

A mixture of 4-biphenylacetic acid (A, 1.50 kg, 7.07 mol) and trifluoroacetic acid (10.5 L, 16.1 kg, 7 vol) was stirred at 21° C. With external cooling, chlorosulfonic acid (3.28 L, 5.76 kg, 49.5 mol, 7 equiv.) was added over 2 h maintaining the internal temperature between 21-25° C. After the addition was completed, the reaction mixture was stirred at 20-21° C. for 20 h. The reaction mixture was divided into two equal portions (2×11.2 kg) and quenched batch-wise as described below.

A solution of water (4 L) and acetic acid (1.30 kg) was cooled to 6° C. With external cooling, the reaction mixture (11.2 kg) was slowly added to the stirred quench solution over 2.5 h maintaining the temperature below 22° C. The mixture was stirred for an additional 30 min and the solid was collected by filtration. The filter-cake was washed with water (3×1.5 L) and dried under suction providing sulfonyl chloride (C) as a wet-cake. This procedure was repeated for the second portion and afforded a combined 6.34 kg of 2-(4′-(chlorosulfonyl)biphenyl-4-yl)acetic acid (C) as a wet-cake. HPLC purity, 94% (by peak area). Mass calculated for C₁₄H₁₁ClO₄S: 310.0, Found: LCMS m/z (%)=311.1 [M+H]⁺ (40), 265.0 (100); ¹H NMR (400 MHz, CDCl₃): δ 8.10 (d, J=8.8 Hz, 2H), 7.80 (d, J=8.8 Hz, 2H), 7.61 (d, J=8.4 Hz, 2H), 7.45 (d, J=8.4 Hz, 2H), 3.75 (s, 2H).

### Example 2

### Synthesis of 2-(4′-(3-Methoxypropylsulfonyl)biphenyl-4-yl)acetic Acid by Alkylation of 2-(4′-(Chlorosulfonyl)biphenyl-4-yl)acetic Acid

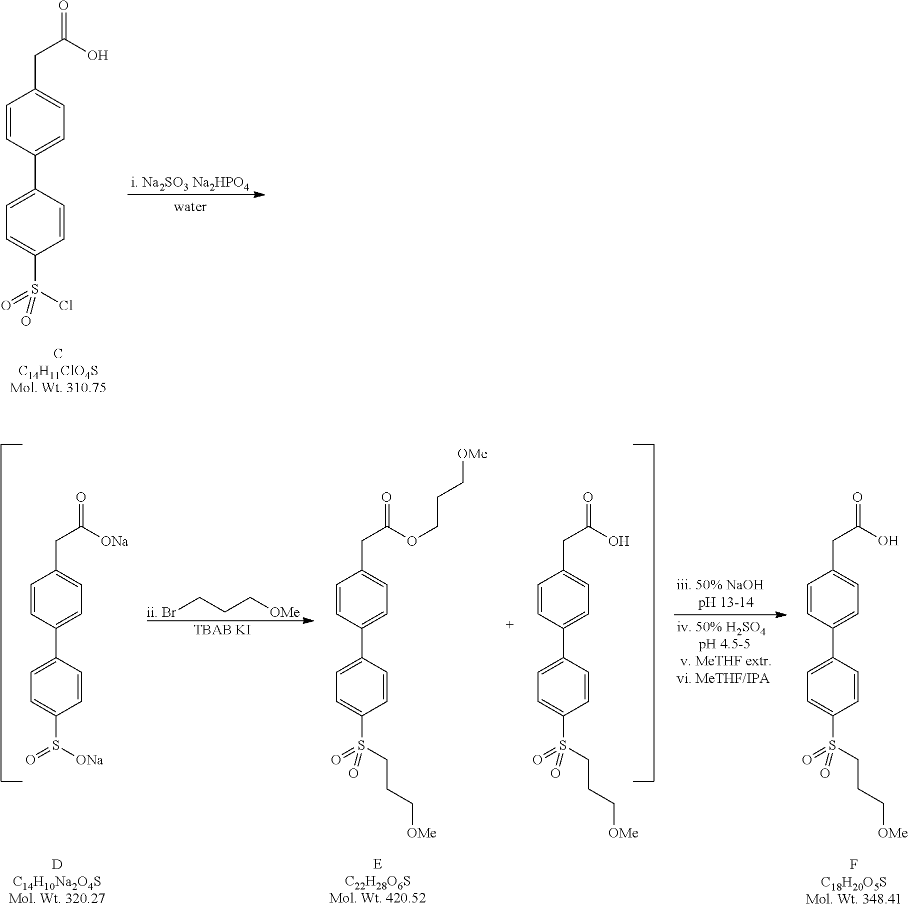

A solution of water (12.0 L), sodium sulfite (1.22 kg, 3.0 equiv.), and sodium phosphate, dibasic (1.14 kg, 2.5 equiv.) was degassed with nitrogen for at least 30 min. The wet-cake containing 2-(4′-(chlorosulfonyl)biphenyl-4-yl)acetic acid (C, 1.00 kg, 3.21 mol) was charged in one portion. After sparging again with nitrogen for at least 10 min, the contents were heated at 60° C. for 1 h.

When the reaction was determined complete, tetrabutylammonium bromide (0.10 kg, 0.10 equiv.) and KI (0.05 kg, 0.10 equiv.) were charged to the reaction solution. The mixture was heated at 70-75° C. and 1-bromo-3-methoxypropane (2.02 kg, 4.10 equiv) over 12 h. The mixture was cooled to ambient temperature and 50 wt % aqueous NaOH solution (1.33 kg) was added; the pH of the reaction solution was adjusted to 13-14. The mixture was heated at 80° C. for at least 1 h. The mixture was cooled to 60° C. and a solution of aqueous H₂SO₄ (50 v/v %, 1.20 kg) was charged adjusting the pH to 4.5-5. The contents were then partitioned with 2-methyltetrahydrofuran (2-MeTHF; 4.3 kg) at 60-65° C. and the biphasic mixture was cooled to 25° C. The phases were separated and the organic phase was washed with water (2.00 kg). The organic phase was concentrated at 40-50° C. under reduced pressure to remove the majority of solvent. The concentrate was diluted with i-PrOH (1.2 kg) and re-concentrated to remove most of the solvent. The concentrate was diluted with i-PrOH (2.36 kg) and heated at 70-80° C. to dissolve the solid. The solution was cooled to 20° C. and aged at 20° C. for at least 2 h. The solid was collected by filtration and the filter-cake was washed with cold i-PrOH (1.37 kg). The filter-cake was dried by suction and then further dried under reduced pressure (30° C./20 torr) to afford 2-(4′-(3-methoxypropylsulfonyl)biphenyl-4-yl)acetic acid (0.896 kg, 80% yield) as an off-white powder. HPLC purity, 98.7% (by peak area). KF: 0.4 wt % H₂O. Mass calculated for: C₁₈H₂₀O₅S: 348.1, Found: LCMS m/z (%)=349.4 [M+H]⁺ (32), 317.1 [M+H−CH₃OH]⁺ (100); ¹H NMR (400 MHz, CDCl3) δ 7.97 (d, J=8.6 Hz, 2H), 7.75 (d, J=8.6 Hz, 2H), 7.60 (d, J=8.3 Hz, 2H), 7.42 (d, J=8.3 Hz, 2H), 3.74 (s, 2H), 3.46 (t, J=6.0 Hz, 2H), 3.29 (s, 3H), 3.22-3.26 (m, 2H), 2.00-2.07 (m, 2H).

### Example 3

### Synthesis of 2-(4′-(3-Methoxypropylsulfonyl)biphenyl-4-yl)ethanol by Reduction of 2-(4′-(3-Methoxypropylsulfonyl)biphenyl-4-yl)acetic Acid

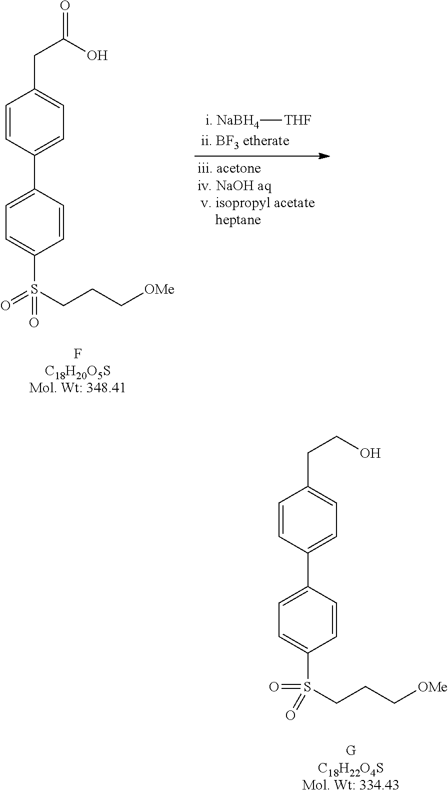

A mixture of 2-(4′-(3-methoxypropylsulfonyl)biphenyl-4-yl)acetic acid (1.00 kg, 2.87 mol) and NaBH₄ (163 g,1.50 equiv.) was diluted with THF (5.42 kg). The mixture was cooled at 5-10° C. and BF₃.OEt₂ (0.62 kg, 1.50 equiv.) was added while maintaining the temperature below 15° C. After the addition was completed, the reaction mixture was agitated at 0-5° C. for an additional 1.5 h. After the reaction was completed, acetone (1.74 kg) was charged and the reaction mixture was heated at 60-65° C. for 2 h. Aqueous NaOH solution (50 wt %, 1.74 kg) was slowly added to the reaction mixture and the contents were heated at 80° C. for 2 h. The mixture was cooled to 20-25° C. and concentrated under reduced pressure to 20% of the original volume. The concentrate was partitioned between water (4.00 kg) and i-PrOAc (8.72 kg), heated at 50° C. for 1 h, and the phases were separated. The organic phase was washed with water (2×3.00 L). The organic phase was concentrated under reduced pressure to about ⅓ volume (3.6 L). The concentrate was heated at 60° C., diluted with heptane (4.00 kg), cooled to 0-5° C., and stirred at 0-5° C. for 2 h. The solid was collected by filtration, dried by suction, and dried further under reduced pressure (45° C./20 torr) to afford 2-(4′-(3-methoxypropylsulfonyl)biphenyl-4-yl)ethanol (0.905 kg, 94%) as an off-white powder. The purity was 99.0 area % by HPLC. KF: 0.19 wt % water. Mass calculated for: C₁₈H₂₂O₄S: 334.1. Found: LCMS m/z (%)=335.5 [M+H]⁺ (58), 303.4 [M+H−CH₃OH]⁺ (100); ¹H NMR (400 MHz, CDCl₃): δ 7.97 (d, J=8.5 Hz, 2H), 7.76 (d, J=8.5 Hz, 2H), 7.57 (d, J=8.2 Hz, 2H), 7.37 (d, J=8.2 Hz, 2H), 3.94 (t, J=6.5 Hz, 2H), 3.45 (t, J=6.0 Hz, 2H), 3.29 (s, 3H), 3.22-3.26 (m, 2H), 2.95 (t, J=6.5 Hz, 2H), 2.00-2.07 (m, 2H), 1.49 (bs, 1H).

### Example 4

### Synthesis of 2-(4′-(3-Methoxypropylsulfonyl)biphenyl-4-yl)ethyl Methanesulfonate by Methylsulfonylation of 2-(4′-(3-Methoxypropylsulfonyl)biphenyl-4-yl)ethanol

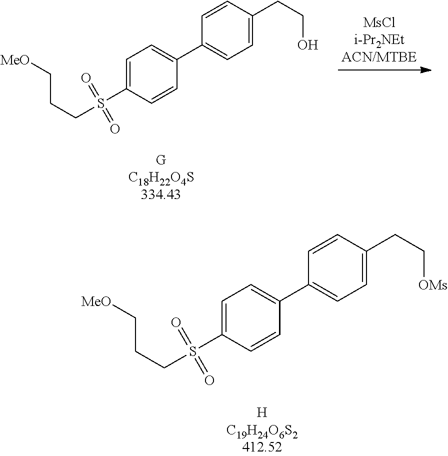

### Method 1

A solution of 2-(4′-(3-methoxypropylsulfonyl)biphenyl-4-yl)ethanol (12.1 kg, 36.2 mol), acetonitrile (ACN, 15.0 kg), methyl t-butyl ether (MTBE, 57 kg), and N,N-diisopropylethylamine (6.68 kg, 1.40 equiv.) was cooled at 0 to 5° C. To the cold solution, MsCl (5.74 kg, 1.40 equiv.) was added over 50 min at a rate to maintain the temperature at 0-5° C. After the addition was completed, the solution was stirred at 0-5° C. for an additional 2 h. The solution was quenched with water (30 kg, 2.5 volumes) while maintaining the temperature from 0-10° C. The temperature of the quenched mixture was raised to 25° C., and the phases were separated. The organic phase was washed with water (30 kg) at 25-30° C. and washed again with water (30 kg) at 35° C., separating the phases after each washing. The organic phase was diluted with methyl t-butyl ether (36 kg) and heated at 55-60° C. for 1 h. The mixture was cooled to 0-5° C. over 2 h and held at 0-5° C. for 1 h. The solid was collected by filtration, the filter-cake was washed with methyl t-butyl ether (19 kg), dried with suction, and further dried at under reduced pressure (45° C./15 torr) to afford the desired 2-(4′-(3-methoxypropylsulfonyl)biphenyl-4-yl)ethyl methanesulfonate (12.4 kg, 82.9%) as a white powder.

Mass calculated for: C₁₉H₂₄O₆S₂: 412.1, Found: LCMS m/z (%)=413.5 [M+H]⁺ (39), 381.2 [M+H−CH₃OH]⁺ (100); 1H NMR (400 MHz, CDCl₃): δ 7.97 (d, J=8.4 Hz, 2H), 7.76 (d, J=8.4 Hz, 2H), 7.59 (d, J=8.2 Hz, 2H), 7.37 (d, J=8.1 Hz, 2H), 4.48 (t, J=6.8 Hz, 2H), 3.45 (t, J=5.9 Hz, 2H), 3.29 (s, 3H), 3.26-3.22 (m, 2H), 3.14 (t, J=6.8 Hz, 2H), 2.94 (s, 3H), 2.06-1.99 (m, 2H).

### Method 2

A solution of 2-(4′-(3-methoxypropylsulfonyl)biphenyl-4-yl)ethanol (200 g, 598 mol), acetonitrile (670 mL) and N,N-diisopropylethylamine (146 mL, 837 mmol) was cooled at 0 to 5° C. MsCl (65.2 mL, 837 mmol) in acetonitrile (130 mL) was added to the cold solution over 30 min at a rate sufficient to maintain the temperature at 0 to 5° C. After the addition was completed, the solution was stirred at 5° C. for an additional 1 h. The reaction was slowly quenched with ice water (2.4 L) while maintaining the temperature from 0 to 5° C. The solid was collected by filtration and the filter cake was washed with water (3×800 mL) and MTBE (2×800 mL) to leave the title compound (242 g, 97%). Purity: 99.1% by HPLC.

### Example 5

### Preparation of (R)-1-{2-[4′-(3-Methoxy-propane-1-sulfonyl)-biphenyl-4-yl]-ethyl}-2-methyl-pyrrolidine and Conversion to the Di-citrate Salt

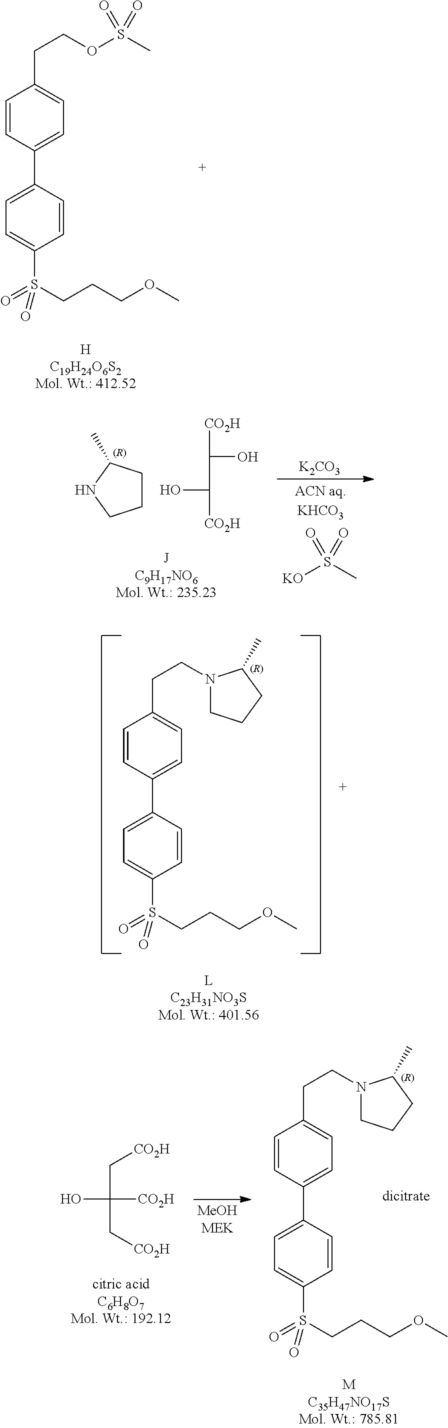

### Method 1

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate (1.019 kg, 2.47 mmol), anhydrous K₂CO₃ (1.024 kg, 3 eq.), (R)-2-methylpyrrolidine L-tartrate (814 g, 1.4 eq.), acetonitrile (8.15 L, 8 volumes), and water (2.86 L, 2.8 volumes) was heated at 70° C. for 24 h. After the reaction was completed, the mixture was concentrated by distillation, under reduced pressure, to remove most of the acetonitrile (7.7 L). The concentrate was partitioned with 2-butanone (methyl ethyl ketone, MEK, 3.05 L, 3 volumes), the resultant phases were separated, and the organic phase was washed with a solution of 20 wt % NaCl in water (3.0 kg). The organic phase was distilled to remove water azeotropically. After 2.5 L of distillate was removed, the concentrate was diluted with 2-butanone (2.5 L).

### Step B: Preparation of (R)-1-{2-[4′-(3-Methoxy-propane-1-sulfonyl)-biphenyl-4-yl]-ethyl}-2-methyl-pyrrolidine Di-citrate

Anhydrous citric acid (1.043 kg, 2.2 eq.) and methanol (3.06 L, 3 volumes) were charged to the organic phase. The mixture was warmed at 60° C. and diluted with 2-butanone (10volumes) while maintaining the temperature between 55-60° C. The mixture was cooled to 0-5° C. over 5 h and held at 0-5° C. for 4 h. The solid was collected by filtration and the filter-cake was washed with 2-butanone (2×1.5 L). The filter-cake was dried with suction and further dried under reduced pressure (45° C./10 torr) to afford the title compound as a white powder (1.642 kg, 85%).

Analytical data from a representative batch: HPLC purity was 99.7 area %. Exact mass calculated for: C₂₃H₃₂NO₃S⁺402.2097, found: LCMS m/z=402.2021 [M+H]⁺; ¹H NMR (400 MHz, DMSO-d₆): δ 10.91 (bs, 6H), 7.95 (s, 4H), 7.76 (d, J=8.2 Hz, 2H), 7.48 (d, J=8.2 Hz, 2H), 3.62-3.56 (m, 1H), 3.54-3.41 (m, 3H), 3.36-3.32 (m, 4H), 3.24-3.15, m, 2H), 3.17 (s, 3H), 3.10-2.96 (m, 2H), 2.61 (dd, J=35.0, 15.2 Hz, 8H), 2.23-2.14 (m, 1H), 1.99-1.90 (m, 2H), 1.82-1.75 (m, 2H), 1.66-1.56 (m, 1H), 1.35 (d, J=6.6 Hz, 3H).

### Method 2

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate (12.2 kg, 29.6 mol), anhydrous K₂CO₃ (12.3 kg, 3 eq.), (R)-2-methylpyrrolidine L-tartrate (9.76 kg, 1.4 eq.), acetonitrile (97.5 L, 8 volumes), and water (34.2 L, 2.8 volumes) was heated at 70-75° C. for 20 h. After the reaction was completed, the mixture was concentrated by distillation, under reduced pressure, to remove most of the acetonitrile. The concentrate was partitioned between 2-butanone (38.7 L, 3 volumes) and additional water (7.7 L, 0.6 volumes). The resultant phases were separated and the organic phase was washed with a solution of 20 wt % NaCl in water (36.8 kg). The organic phase was clarified by recirculation through in-line filters and diluted with 2-butanone (7.8 L, 0.6 volumes).

### Step B: Preparation of (R)-1-{2-[4′-(3-Methoxy-propane-1-sulfonyl)-biphenyl-4-yl]-ethyl}-2-methyl-pyrrolidine Di-citrate

A previously prepared solution of anhydrous citric acid (12.4 kg, 2.2 eq.) and methanol (36.7 L, 3 volumes) was charged to the organic phase. The mixture was warmed at 60-65° C., cooled at 50-55° C., and diluted with 2-butanone (121 L, 10 volumes) while maintaining the temperature between 55-60° C. The reactor contents were warmed to 62° C. and then cooled to 37° C. over 1 h. The temperature was rapidly cooled to 10° C. to induce crystallization. The resultant mixture was further cooled to 0-5° C. and aged for 9 h. An attempt to collect the solid by filtration failed due to poor filtration properties. The portion of wet cake that was collected was redissolved in hot MeOH (90 L, 7 volumes) and added back to the unfiltered mixture. The mixture was distilled under reduced pressure and recharged with 2-butanone until the desired 20 wt % methanol in 2-butanone (16.5 volumes) was achieved. After the solvent ratio and volume were adjusted back to their desired values the reactor contents were cooled to 30° C., seeded, and aged at 30° C. The contents were further cooled to and aged at 0-5° C. The solid was collected by filtration, the filter-cake was washed with 2-butanone (4×2 volumes), and dried under reduced pressure with heat and a nitrogen sweep to afford a 1^{st} crop of the title compound (12.6 kg, 54.0%) as a white powder containing a low level of mono-methyl citrate. The mother liquor and washings were combined and concentrated under reduced pressure to 12 wt % methanol in 2-butanone (˜6 volumes). After cooling to and aging at 0-5° C., the solid was collected by filtration, washed with 2-butanone (3×1 volume), and dried under reduced pressure at 50° C. to afford a second crop (4.12 kg, 17.7%) of the title compound as a white powder containing a low level of mono-methyl citrate.

### Step C: Purification of (R)-1-{2-[4′-(3-Methoxy-propane-1-sulfonyl)-biphenyl-4-yl]-ethyl}-2-methyl-pyrrolidine Di-citrate

A portion of the crude (R)-1-{2-[4′-(3-methoxy-propane-1-sulfonyl)-biphenyl-2-yl]-ethyl}-2-methyl-pyrrolidine di-citrate (200 g, 0.485 mol) was slurried with anhydrous citric acid (4.89 g, 0.10 eq.) in water (60 mL, 0.3 volumes) and acetonitrile (1.94 L, 9.7 volumes) and heated at 60-65° C. for 48 h. The slurry was cooled to 0-5° C. over 2.5 h, aged at 0-5° C. for 2 h, and the solid was collected by filtration. The filter-cake was washed with acetonitrile (800 mL, 4 volumes), allowed to dry by suction, and dried further under reduced pressure at 45-50° C. to afford the title compound as a white, crystalline solid (188.4 g, 94.2%). HPLC analysis of the counter ions showed 99.5 area % citric acid and 0.39 area % mono-methyl citrate. HPLC analysis of the parent showed a purity of 99.8 area %.

### Method 3

### Step A: Preparation of (R)-1-{2-[4′-(3-Methoxy-propane-1-sulfonyl)-biphenyl-4-yl]-ethyl}-2-methyl-pyrrolidine

A biphasic mixture of 2-(4′-(3-methoxypropylsulfonyl)biphenyl-4-yl)ethyl methanesulfonate, anhydrous K₂CO₃ (3 eq.), (R)-2-methylpyrrolidine L-tartrate (1.4 eq.), acetonitrile (8 volumes), and water (2.8 volumes) is heated at 70° C. for 24 h. After the reaction is completed, the mixture is concentrated by distillation, under reduced pressure, to remove most of the acetonitrile. The concentrate is diluted with a water-immiscible organic solvent (e.g. ethyl acetate or methyl t-butyl ether; 3 volumes), the resultant phases are separated, and the organic phase is washed with water (3 volumes). The organic phase is concentrated by distillation to remove most of the solvent and acetonitrile (9.7 volumes) is added.

### Step B: Preparation of (R)-1-{2-[4′-(3-Methoxy-propane-1-sulfonyl)-biphenyl-4-yl]-ethyl}-2-methyl-pyrrolidine Di-citrate

Anhydrous citric acid (2.2 eq.) and water (0.3 volumes) are charged to the organic phase. The resultant mixture is warmed at 60° C. and heated at 60-65° C. for 12-48 h. The slurry is cooled to 0-5° C. over 2-4 h, aged at 0-5° C. for 2 h, and the solid is collected by filtration. The filter-cake is washed with acetonitrile (3×4 volumes), allowed to dry by suction, and dried further under reduced pressure at 40-50° C. to afford the title compound.

### Method 4

### Preparation of (R)-1-{2-[4′-(3-Methoxy-propane-1-sulfonyl)-biphenyl-4-yl]-ethyl}-2-methyl-pyrrolidine Di-citrate

2-(4′-(3-Methoxypropylsulfonyl)biphenyl-4-yl)ethyl methanesulfonate (200 g, 485 mmol) and (R)-2-methylpyrrolidine L-tartrate (160 g, 679 mmol) were charged into a 4 L vertical reactor equipped with a thermocouple, a N₂ inlet and an overhead stirrer. 2-Butanone (4 volumes) and aqueous NaOH (273 mL, 2182 mmol) were added. The biphasic system was stirred and heated to reflux (74° C., internal). The reaction mixture was allowed to stir at this temperature overnight. The reaction mixture was then cooled down to 20° C. over 1 h and allowed to stir at that temperature for approximately 64 hours. Water (2 volumes) and 2-butanone (2 volumes) were added and the mixture was allowed to stir until all solids had dissolved. The phases were allowed to separate and the aqueous phase was removed. The organic phase was washed with water (2×1 volume) and then concentrated by vacuum distillation (1 L of distillate was collected). 2-Butanone (6 volumes) was added to the residue and again the mixture was concentrated by vacuum distillation (1.3 L of distillate was collected). 2-Butanone (530 mL) was added to the residue, which was filtered for clarification, rinsing with more 2-butanone (418 mL), to give an orange-colored solution. This solution was heated to 70° C. and citric acid (205 g, 1067 mmol) in water (82.3 mL) also at 70° C., was added. The mixture was cooled to 60° C. and allowed to stir at that temperature over night. 2-Butanone (1.72 L) was added at a rate sufficient to maintain an internal temperature of 58 to 60° C. and then the mixture was allowed to stir at 60° C. for 1.5 h. The mixture was then cooled to 0° C. over 105 min and stirred at that temperature for 1 h. The mixture was filtered and the filter cake was slurry-rinsed first with 2-butanone:water (98:2, 3 volumes), and then with 2-butanone (2×2 volumes). The solid was dried in a vacuum oven at 40° C. overnight to leave the title compound (349 g, 92%).

### Example 6a

### Preparation of (R)-1-{2-[4′-(3-Methoxy-propane-1-sulfonyl)-biphenyl-4-yl]-ethyl}-2-methyl-pyrrolidine Maleate

(R)-1-{2-[4′-(3-Methoxy-propane-1-sulfonyl)-biphenyl-4-yl]-ethyl}-2-methyl-pyrrolidine free base (1.6 g) was dissolved in acetone (20 mL). Addition of maleic acid (about 0.015 mL of a 4.15 M aqueous solution) to an aliquot of the acetone solution of (R)-1-{2-[4′-(3-methoxy-propane-1-sulfonyl)-biphenyl-4-yl]-ethyl}-2-methyl-pyrrolidine free base (0.31 mL) gave a solution which was evaporated to dryness. To the resulting thick oil was added IPA (about 0.3 mL) before heating briefly to about 50° C. in a ReactiTherm to get the oil into solution. The solution was allowed to cool down and stir at room temperature overnight. The precipitate was collected by centrifuge filtration and air dried. NMR (400 MHz, DMSO-d₆) δ ppm 1.40 (d, J=6.27 Hz, 3H), 1.58-1.68 (m, 1H), 1.79-1.86 (m, 2H), 1.90-2.07 (m, 1H), 2.99-3.15 (m, 2H), 3.20 (s, 3H), 3.23-3.42 (m, 7H), 3.45-3.70 (m, 3H), 6.05 (s, 4H), 7.51 (d, J=8.16 Hz, 2H), 7.79 (d, J=8.28 Hz, 2H), 7.99 (s, 4H).

### Example 6b

### Preparation of (R)-1-{2-[4′-(3-Methoxy-propane-1-sulfonyl)-biphenyl-4-yl]-ethyl}-2-methyl-pyrrolidine Maleate

(R)-1-{2-[4′-(3-Methoxy-propane-1-sulfonyl)-biphenyl-4-yl]-ethyl}-2-methyl-pyrrolidine free base (1.6 g) was dissolved in acetone (20 mL). Addition of maleic acid (about 0.015 mL of a 4.15 M aqueous solution) to an aliquot of the acetone solution of (R)-1-{2-[4′-(3-methoxy-propane-1-sulfonyl)-biphenyl-4-yl]-ethyl}-2-methyl-pyrrolidine free base (0.31 mL) gave a solution which was evaporated to dryness. To the resulting thick oil was added IPA (about 0.3 mL) before heating briefly to about 50° C. in a ReactiTherm to get the oil into solution. The solution was allowed to cool down and stir at room temperature overnight. Precipitation occurred during cooling, or optionally a maleate seed crystal can be added to assist in precipitation. The precipitate was collected by centrifuge filtration and air dried to provide (R)-1-{2-[4′-(3-methoxy-propane-1-sulfonyl)-biphenyl-4-yl]-ethyl}-2-methyl-pyrrolidine maleate.

### Example 7

### Preparation of (R)-1-{2-[4′-(3-Methoxy-propane-1-sulfonyl)-biphenyl-4-yl]-ethyl}-2-methyl-pyrrolidine Hydrochloride

(R)-1-{2-[4′-(3-Methoxy-propane-1-sulfonyl)-biphenyl-4-yl]-ethyl}-2-methyl-pyrrolidine free base was obtained by neutralization of the (R)-1-{2-[4′-(3-methoxy-propane-1-sulfonyl)-biphenyl-4-yl]-ethyl}-2-methyl-pyrrolidine di-citrate (2.0 g) with 0.5 N aqueous solution of NaOH (25 mL). After extraction with isopropyl acetate, the organics were separated, washed with water, dried over MgSO₄, filtered and concentrated to afford a colorless viscous oil. The oil (0.2 g to 0.5 g) was dissolved in diethyl ether (20 mL to 50 mL) before an ethereal solution of 1 M HCl was added (to pH 1) to afford a sticky, waxy semi-solid. After overnight stirring of the semi-solid in a closed system, a free flowing white solid was obtained, filtered under a N₂ blanket and rinsed with diethyl ether.

All references cited herein are incorporated by reference. A number of embodiments of the invention have been described. Nevertheless, it will be understood that various modifications may be made without departing from the spirit and scope of the invention. Accordingly, other embodiments are within the scope of the following claims.

1. A process for preparing a compound according to formula I: 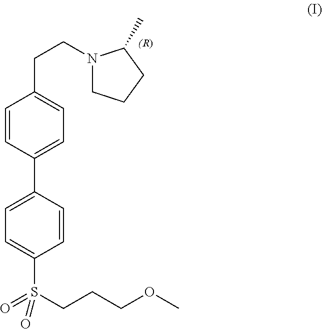
or a salt thereof, comprising:
(a) chlorosulfonating a compound according to formula IX: 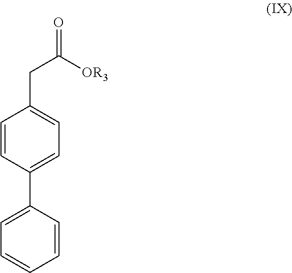
or a salt thereof, to form a compound according to formula VIII: 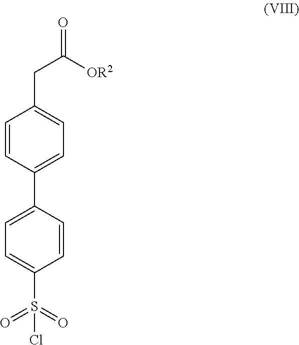
or a salt thereof;
(b) reducing said compound according to formula VIII or a salt thereof, to form a compound according to formula VI: 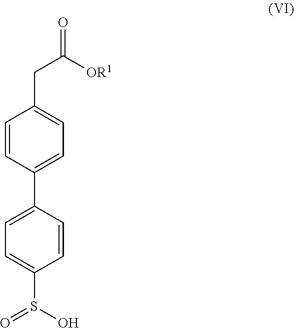
or a salt thereof;
(c) reacting said compound according to formula VI or salt thereof, with a compound according to formula VII: 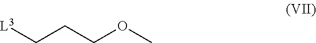
under conditions sufficient to effect displacement of the leaving group L³ of said compound according to formula VII by the sulfinate group of said compound according to formula VI to form a compound according to formula V: 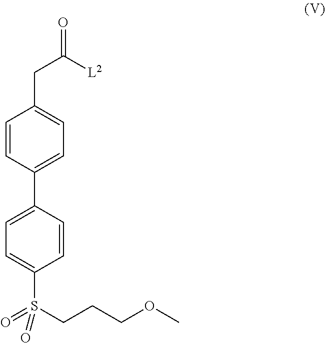
(d) reducing said compound according to formula V to form a compound according to formula IV: 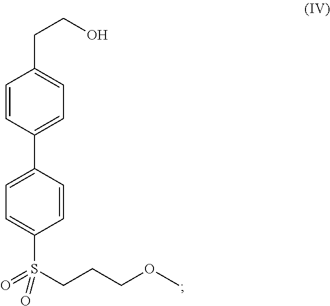
(e) reacting said compound according to formula IV to form a compound according to formula II: 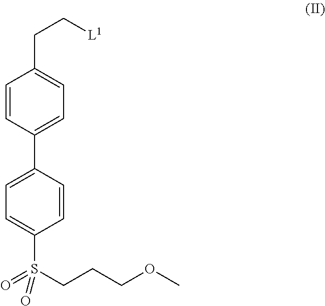
under conditions sufficient to effect conversion of the hydroxyl group of said compound according to formula IV to form the leaving group L¹ of said compound according to formula II; and
(f) reacting said compound according to formula II with a compound according to formula III: 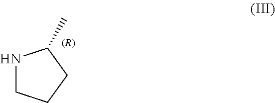
or a salt thereof, under conditions sufficient to effect displacement of the leaving group L¹ of said compound according to formula II by the amino group of said compound according to formula III to form said compound according to formula I, or a salt thereof;
wherein:
L¹ is a suitable leaving group selected from iodide and a sulfonate ester group;
L² is hydroxyl, or a salt of the hydroxyl, or L² is C₁-C₆ alkoxy;
L³ is a suitable leaving group;
R¹ is hydrogen or C₁-C₆ alkyl;
R² is hydrogen or C₁-C₆ alkyl; and
R³ is hydrogen or C₁-C₆ alkyl.

2. A process for preparing a compound according to formula I: 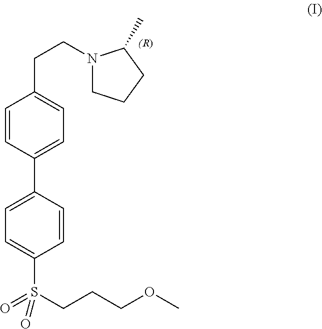
or a salt thereof, comprising reacting a compound according to formula II: 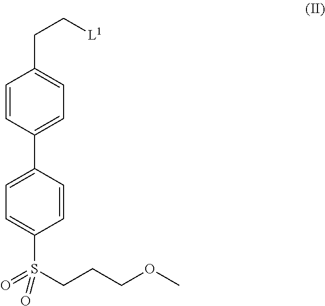
wherein L¹ is a suitable leaving group selected from iodide and a sulfonate ester group with a compound according to formula III: 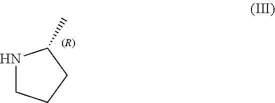
or a salt thereof, under conditions sufficient to effect displacement of the leaving group L¹ of the compound according to formula II by the amino group of the compound according to formula III to form the compound according to formula I, or a salt thereof.

3. A process according to claim 2, wherein L¹ is a methanesulfonate ester group.

4. A process according to claim 2, wherein the reacting is performed in the presence of a suitable base.

5. A process according to claim 4, wherein the base is potassium carbonate.

6. A process according to claim 4, wherein the base is sodium hydroxide.

7. A process according to claim 2, wherein the reacting is performed in the presence of an aprotic solvent.

8. A process according to claim 7, wherein the aprotic solvent comprises 2-butanone.

9. A process according to claim 7, wherein the aprotic solvent comprises acetonitrile.

10. A process according to claim 7, wherein the reacting is performed in the presence of water.

11. A process according to claim 2, wherein the reacting is performed at a temperature in the range from about 30° C. to about 120° C.

12. A process according to claim 2, further comprising reacting the compound according to formula I with an acid and isolating a salt of the compound according to formula I.

13. A process according to claim 12, wherein the salt is a di-citrate.

14. A process for preparing a compound according to formula II: 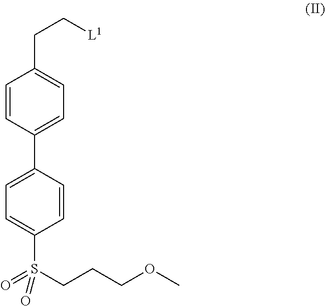
wherein L¹ is a leaving group selected from iodide and a sulfonate ester group, comprising reacting a compound according to formula IV: 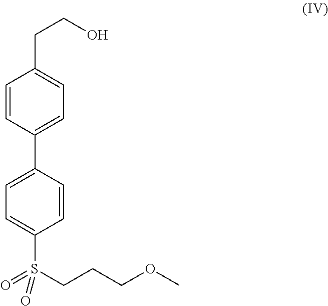
under conditions sufficient to effect conversion of the hydroxyl group to form the leaving group L¹ of the compound according to formula II.

15. A process according to claim 14, wherein L¹ is a methanesulfonate ester group, and the compound according to formula II is prepared by reacting the compound according to formula IV with methanesulfonyl chloride.

16. A process according to claim 14, wherein the reaction to form the compound according to formula II is performed in a reaction mixture comprising an aprotic solvent.

17. A process according to claim 16, wherein the solvent comprises a C₂-C₄ alkanonitrile.

18. A process according to claim 16, wherein the solvent comprises a mixture of an aliphatic ether and a C₂-C₄ alkanonitrile.

19. A process according to claim 14, wherein the reaction to form the compound according to formula II is performed in a reaction mixture comprising a base.

20. A process according to claim 14, wherein the reaction to form the compound according to formula II is performed at a temperature in the range from about −20° C. to about 20° C.

21. A process for preparing a compound according to formula IV: 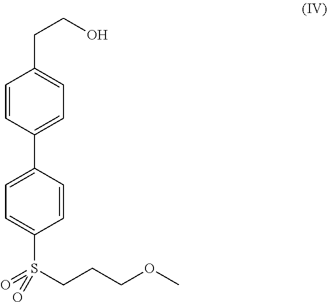
comprising reducing a compound according to formula V: 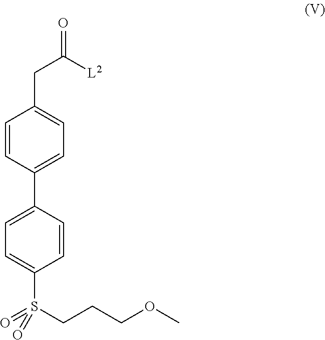
wherein L² is hydroxyl, or a salt of the hydroxyl, or L² is C₁-C₆ alkoxy.

22. A process according to claim 21, wherein L² is hydroxyl or a salt of the hydroxyl, wherein reducing the compound according to formula V is performed by reacting the compound with an alkali metal borohydride in the presence of boron trifluoride.

23. A process according to claim 21, wherein reducing the compound according to formula V is performed in an aliphatic ether solvent.

24. A process according to claim 21, wherein reducing the compound according to formula V is performed at a temperature in the range from about −20° C. to about 30° C.

25. A process for preparing a compound according to formula V: 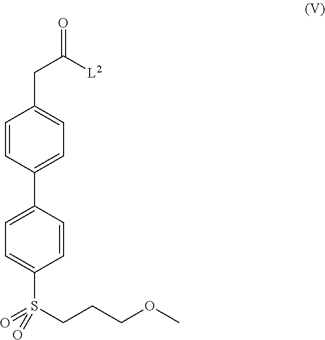
wherein L² is hydroxyl, or a salt of the hydroxyl, or L² is C₁-C₆ alkoxy, comprising reacting a compound according to formula VI: 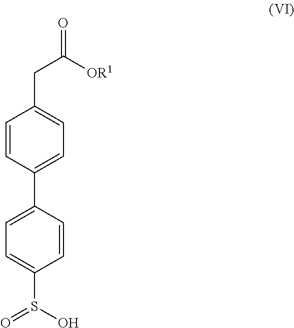
or salt thereof, wherein R¹ is hydrogen or C₁-C₆ alkyl, with a compound according to formula VII: 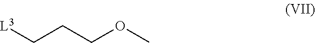
wherein L³ is a suitable leaving group, under conditions sufficient to effect displacement of the leaving group L³ of the compound according to formula VII by the sulfinate group of the compound according to formula VI.

26. A process according to claim 25, wherein L³ is chloride, bromide, iodide, or a sulfonate ester group.

27. A process according to claim 25, wherein R¹ is hydrogen.

28. A process according to claim 25, wherein the reaction forming the compound according to formula V is performed using an alkali metal salt of the compound according to formula VI.

29. A process according to claim 25, wherein the reaction forming the compound according to formula V is performed in the presence of a catalyst.

30. A process according to claim 25, wherein the reaction forming the compound according to formula V is performed at a temperature in the range from about 30° C. to about 120° C.

31. A process for preparing a compound according to formula VI: 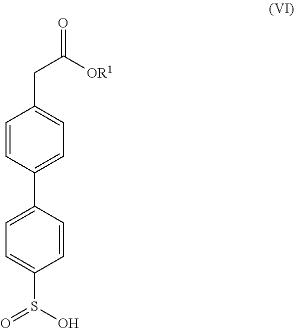
or a salt thereof, wherein R¹ is hydrogen or C₁-C₆ alkyl, comprising reducing a compound according to formula VIII: 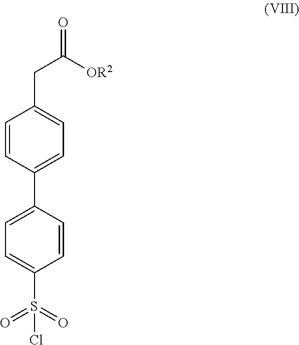
or a salt thereof, wherein R² is hydrogen or C₁-C₆ alkyl.

32. A process according to claim 31, wherein R² is hydrogen.

33. A process according to claim 31, wherein the reduction of the compound according to formula VIII is performed in a solution comprising water.

34. A process according to any one of claims 31, wherein the reduction of the compound according to formula VIII is performed at a temperature in the range from about 40° C. to about 100° C.

35. A process for preparing a compound according to formula VIII: 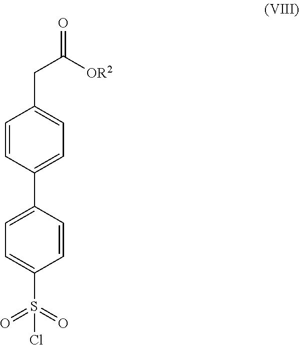
or a salt thereof, wherein R² is hydrogen or C₁-C₆ alkyl, comprising chlorosulfonating a compound according to formula IX: 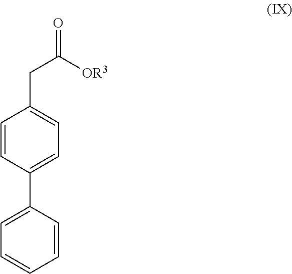
or a salt thereof, wherein R³ is hydrogen or C₁-C₆ alkyl.

36. A process according to claim 35, wherein R³ is hydrogen.

37. A process according to claim 35, wherein the chlorosulfonation reaction is performed in a carboxylic acid solvent.

38. A process according to claim 35, wherein the chlorosulfonation reaction is performed at a temperature in the range from about 0° C. to about 40° C.

39. A process for preparing a citrate salt of a compound according to formula I: 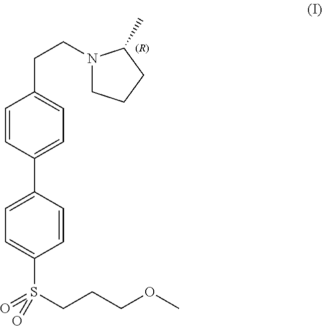
comprising reacting a compound according to formula I with citric acid in a solvent other than acetonitrile.

40. A process according to claim 39, wherein the salt is a di-citrate.

41. A process according to claim 39, wherein the solvent comprises a C₃-C₅ alkanone.

42. A compound according to claim 51 of formula XI: 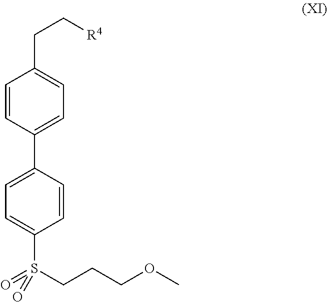
wherein R⁴ is iodide, hydroxyl, or a sulfonate ester.

43. A compound according to claim 42, wherein R⁴ is hydroxyl.

44. A compound according to claim 42, wherein R⁴ is a methanesulfonate ester group.

45. A compound according to claim 51 of formula V: 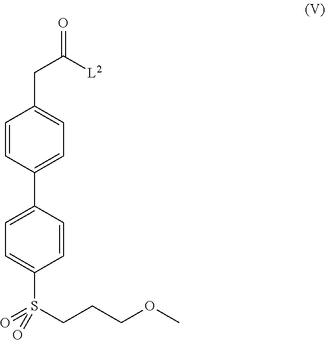
wherein L² is hydroxyl, or a salt of the hydroxyl, or L² is C₁-C₆ alkoxy.

46. A compound according to claim 45, wherein L² is hydroxyl, or a salt of such a compound.

47. A compound according to claim 51 of formula VI: 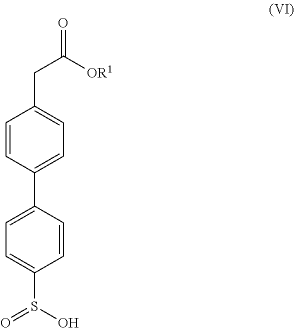
or a salt thereof, wherein R¹ is hydrogen or C₁-C₆ alkyl.

48. A compound according to claim 47, wherein R¹ is hydrogen, or a salt of such a compound.

49. A compound according to claim 51 of formula VIII: 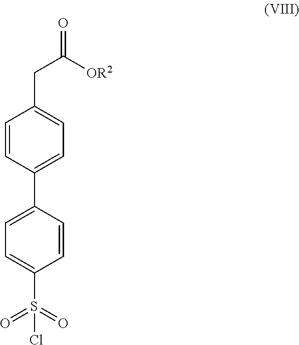
or a salt thereof, wherein R² is hydrogen or C₁-C₆ alkyl.

50. A compound according to claim 49, wherein R² is hydrogen, or a salt of such a compound.

51. A compound selected from compounds of the formulae (V), (VI), (VIII) and (XI): 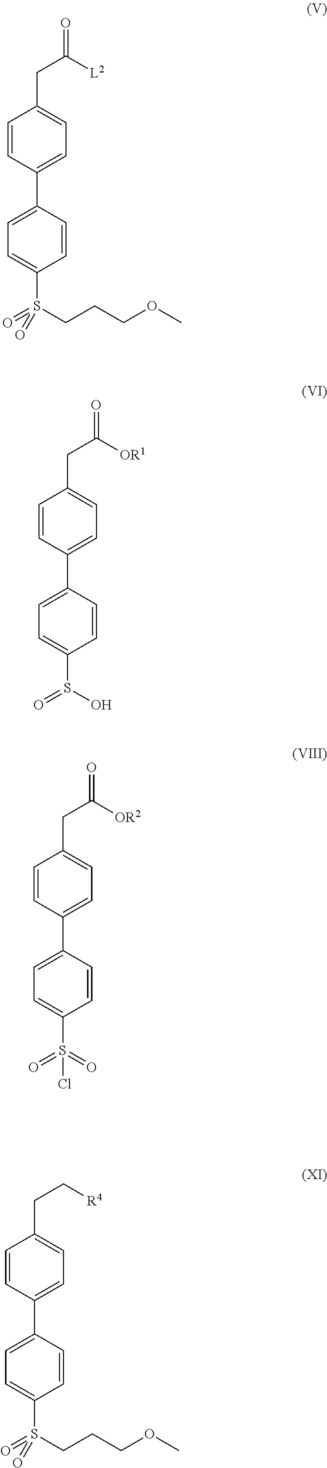
or a salt of such a compound of formula (V), (VI) or (VIII), wherein:
L² is hydroxyl or C₁-C₆ alkoxy;
R¹ is hydrogen or C₁-C₆ alkyl;
R² is hydrogen or C₁-C₆ alkyl; and
R⁴ is iodide, hydroxyl, or a sulfonate ester.
